(19) United States
(12) Patent Application Publication
(10) Pub. No.: US 2004/0248148 A1
(43) Pub. Date: Dec. 9, 2004

(54) Ultrasensitive detection of pathogenic microbes

(76) Inventors: Paul A. Horgen, Mississauga (CA); Rebecca A. Guy, Toronto (CA); Inge V. Tamm, Toronto (CA)

Correspondence Address: SYNNESTVEDT & LECHNER, LLP, 2600 ARAMARK TOWER, 1101 MARKET STREET, PHILADELPHIA, PA, 191072950

(21) Appl. No.: 10/723,125

(22) Filed: Nov. 26, 2003

Related U.S. Application Data
(60) Provisional application No. 60/428,914, filed on Nov. 26, 2002.

(51) Int. Cl. C12Q 1/68; C12P 19/34

(52) U.S. Cl. 435/6; 435/91.2

(57) ABSTRACT

The present invention describes a 5′ nuclease real-time polymerase chain reaction (PCR) approach for the quantification of total coliforms, E. coli, toxigenic E. coli O157:H7, toxigenic M. aeruginosa (microcystin hepatotoxins), Giardia lamblia, and Cryptosporidium parvum, based on the specific identified primer and probe sequences from the lacZ (E. coli), eaeA (E. coli O157:H7), mcyA (M. aeruginosa), β-giardin (G. lamblia), and COWP (C. parvum) genes respectively. The invention allows for the detection of all of the aforementioned microbes, with or without culture enrichments, utilizing a 5′ nuclease PCR approach. The invention also provides primer and probe sequences useful to produce detectable amplicons, by any amplification method, which are diagnostic for such organisms.

### CROSS-REFERENCE TO RELATED APPLICATIONS

This application is based on and claims priority to U.S. Provisional Application No. 60/428,914, filed Nov. 26, 2002, fully incorporated herein by reference.

### FIELD OF THE INVENTION

The present invention relates to improved methods and reagents for detecting the presence of pathogenic microbes in water and clinical samples.

### BACKGROUND OF THE INVENTION

As human population density increases as a result of urban growth, and animal population densities increase from intensive agri-business practices, the pressures on water resources can rise dramatically. Pollution in the form of sewage from human populations, or from livestock in agricultural operations, can lead to elevated levels of microbial contamination in drinking water, irrigation water and ground water, resulting in pathogen contamination of food and recreational water resources. The coliforms including E. coli cause a variety of ailments in humans and domesticated animals, most noticeably urinary tract infections, gastroenteritis, and selected skin disorders.

Traditionally coliforms have been detected and quantified by enzymatic and culturing methods such as the multiple-tube fermentation (MTF) technique to yield most probable number (MPN) or by membrane filtration (MF) and culturing techniques (APHA, 1995; Rompré et al., 2002). Among the drawbacks of these traditional methods is the detection of false positives and the need for further confirmative tests and the long time (on the order of days) and labour required to conduct these tests (Rompré et al., 2002). With culture-based techniques there is also the potential risk of not detecting cells that are metabolically active, but not culturable (viable but not culturable; VBNC). PCR is an efficient method for detection of VBNC cells (Tamanai-Shacoori et al., 1996). PCR-based detection methods can therefore overcome false negatives obtained with culture-based detection methods, and can overcome false positives from some tests due to the sequence-based specificity of PCR testing.

Endpoint PCR has been established as a qualitative method to measure the presence or absence of any given pathogen, including coliforms and has been applied to this problem in the early 1990s (Bej et al. 1990a, 1990b, 1991a, 1991b). A number of gene probes were successful in the studies conducted by Bej et al., including lacZ (total coliforms), uidA (E. coli), and lamB (E. coli, Salmonella, Shigella), and results indicated that the PCR methodologies were as good as, or even more reliable than plate counts or defined substrate methods (Bej et al. 1990a, 1990b). These approaches are reliable, but they are still more time consuming and qualitative in nature than the quantitative measurements that can be obtained with the application of 5′ nuclease PCR to the science of microbial water quality testing.

E. coli O157:H7, EHEC (enterohaemorragic E. coli) is an important water- and foodborn pathogen that can cause a variety of human diseases (Karmali, 1989; Willshaw et al., 1994). It is differentiated from resident microflora by specific biochemical characteristics, such as the inability to ferment sorbitol in 24 hr (Farmer et al., 1985) and the lack of β-glucuronidase activity (Doyle and Schoeni, 1984). Injured or stressed bacteria may not grow on selective media or may not express the antigen required for immunological detection. Immunological methods rely on the specific binding of an antibody to an antigen, for example the interaction of antigens such as lipopolysaccharide (LPS) or Shiga-like toxins (SLTs) with specific antibodies. Conventional and immunological methods are sensitive and permit low numbers of bacteria (˜10³ cellsml⁻¹) to be detected in complex sample matrices. However, the immunological methods do not distinguish between live or dead cells and conventional cultural and immunological methods are often not appropriate for detection of injured or stressed bacteria. E. coli O157:H7 is often present at very low levels, masked by a high population of resident microflora, making the pathogen difficult to detect and subsequently distinguish phenotypically.

There are numerous virulence markers in EHEC (enterohaemorratgic E. coli), they include SLTs (Acheson, 2000), intimin, hemolysin, and the locus of enterocyte effacement (Feng et al., 2001a). Food-borne illnesses have occurred with isolates that possess all or only a few of these markers (Feng et al., 2001b). EHEC strains containing slt1 and slt2 have been isolated from patients with hemorrhagic colitis, studies have shown that strains possessing only slt2 are more frequently associated with human disease complications (Restino et al., 1996). E. coli possessing slts are often referred to as Shiga toxin-producing E. coli (STEC). The eaeA gene has been shown to be necessary for the production of attaching and effacing lesions that are a characteristic of enteropathogenic E. coli (EPEC) (Jerse et al., 1990). The slt1, slt2 and the eae genes have been cloned and sequenced (Jackson et al., 1987; Yu and Kaper, 1992) and the characterization of these virulence factors has led to a better understanding of the pathogenesis of diarrheal diseases caused by these organisms, providing a new dimension to their identification. The slt genes and the eaeA gene have been used for detection with genetic probes and by PCR (Frantamico et al., 1995; Deng and Frantamico, 1996; Germani et al., 1997; Meng et al., 1997). Other genes used for the identification of E. coli O157:H7 by PCR assays include stx1, stx2 (Gannon et al., 1992), eae (Schmidt et al., 1994), rfbE (Desmarchelier et al., 1998) and fliC (Fields et al., 1997; Gannon et al., 1997). Endpoint PCR amplification of eaeA was first reported as a diagnostic tool for the detection of toxigenic E. coli O157:H7 by (Gannon et al., 1993). EaeA encodes intimin, a 97 kDa outer membrane protein (Louie et al., 1993). The 5′ end of the eaeA gene (first 2200 bases) is 97% homologous among EPEC, whereas the last 800 bp of the 3′ end are variable among the strains (Beebakhee et al., 1992; Louie et al., 1994). Applied Biosystems Inc. (ABI) has designed a 5′ nuclease PCR-based diagnostic kit for detection of pathogenic E. coli O157:H7 that will produce plus/minus results with respect to contamination (ABI, 2000). The gene target for this kit is a region of unknown function upstream of the eaeA gene. 5′ nuclease PCR and multiplex endpoint PCR have been used for the detection of E. coli O157:H7 in meat with various regions of the eaeA gene (Oberst et al., 1998; Call et al., 2001). The 3′ end of the eaeA gene was targeted for the detection of E. coli O157:H7 in beef using endpoint PCR (Sharma et al., 1999; Uyttendaele et al., 1999). Many PCR-based detection techniques use the stx1 and stx2 genes, for detecting E. coli O157:H7 (Jothikumar and Griffiths, 2002). However, not all strains of this pathogen have both or either of these genes (Karch et al., 1996; Kim et al., 1998; Feng et al., 2001a). Moreover exploiting multiplex PCR protocols to amplify different genes encoding the virulence factors, with different specific primers, could be a good predictor of the pathogenic potential of E. coli strains.

Polymerase chain reaction-based assays are specific, can be extremely sensitive and results are obtained in a few hours. However, they detect chromosomal gene sequences which can be present in viable and dead cells and, therefore, no determination can be made concerning the presence of only viable cells in a sample (Josephson et al., 1993; Masters et al., 1994). This is a decided disadvantage of PCR-based methods. Several options are available to eliminate the risk of detecting nucleic acid from non-viable cells by PCR, such as reverse-transcription of sample isolated RNA (RNA is less stable than DNA and would be indicative of viable cells in the sample). Several types of RNA are produced in bacterial cells, including ribosomal RNA (rRNA) and messenger RNA (mRNA). rRNA is a universal constituent of bacterial ribosomes and is present in high copy numbers but, similar to DNA, rRNA can persist for an extended period in dead cells (Uyttendaele et al., 1997; McKillip et al., 1998). Messenger RNA is considered a more appropriate target as an indicator of viability since most mRNA species have a short half-life of only a few minutes (Kushner, 1996).

A recent study (Yaron and Mathews, 2002) examined the expression of seven genes of E. coli O157:H7 (rfbE, fliC, stx1, stx2, mobA, eaeA and hly) under a range of conditions to determine a suitable mRNA target(s) for reverse transcriptase (RT)-PCR amplification. Detection based on PCR amplification of these genes has been reported previously (Schmidt et al., 1994; Fields et al., 1997; Desmarchelier etal., 1998). The expression of genes and stability of mRNA were evaluated for samples collected under typical growth conditions, prior to and after thermal treatment of 121° C. for 15 min and 60° C. for 20 min and in cells from a sample (suspension of bacteria in water) which decreased to an undetectable level (<0.1 cfu ml⁻¹) as determined by plate count but contained viable cells based on cytological analysis. The results of RT-PCR amplification indicate that, in most cases, the rfbE gene can be used for detection of viable E. coli O157:H7.

Microcystin-producing cyanobacteria are also a serious threat to both animal and human health due to the toxicity of non-ribosomally produced proteins. This toxin is encoded by the polycistronic microcystin synthetase operon (Nishizawa et al., 1999, 2000). Microcystin phycotoxins, are one of the most common natural biotoxins in fresh as well as marine waters (Andersen et al., 1993; Codd, 1994, 1995; Bury et al., 1997; Sivonen and Jones, 1999). Microcystin is a cyclic heptapeptide produced by toxic strains of M. aeruginosa, as well as species of Anabaena, Nostoc, and Oscillatoria (Codd, 1995; Sivonen and Jones, 1999). This peptide is hepatotoxic and acts by inhibiting protein phosphatases type 1 and 2A, which are tumor suppressors (Sivonen and Jones, 1999), and it has been directly associated with the production of liver cancer in humans, fish, and livestock. Microcystin toxin levels are increasing in the Great Lakes as a result of a number of factors including selective filtration by zebra mussels (Vanderploeg et al., 2001).

There are a number of different methodologies currently in use to detect the toxin. These include high-performance liquid chromatography (HPLC), mass spectrometry, ELISAs (Chu et al., 1989), and other enzyme-based methods, which can be applied to water, cyanobacterial scums and clinical material (Codd et al., 1994). ELISAs offer a relatively narrow range in which microcystin can be quantitated in samples. Relative to ELISAs, HPLC is a relatively time-consuming process. Neither of these assays can distinguish between the toxic and non-toxic variant of microcystis. None of the above methods are capable of detecting the presence of the pathogen itself, as we are able to with real-time PCR. The ability to detect the toxin-producing pathogen itself, rather than the toxin would allow pro-active control of microcystin-producing cyanobacteria in water. Competitive endpoint PCR has been used for the quantification of Microcystis in water by amplification of the 16SrDNA sequence, and subsequently didioxy fluorescein cycle labeled, followed by chromogenic detection (Rudi et al., 1998).

G. lamblia (known also as G. intestinalis and G. duodenalis) and C. parvum are protozoan parasites that cause severe diarrheal illness in human hosts. Symptoms include profuse watery diarrhea, nausea, cramps, malabsorption and last for 2 or more weeks (Vesy and Peterson, 1999; Chen et al., 2002). While infections are usually self-limiting in immunocompetant individuals, chronic infections can be life-threatening in immunocompromised individuals, such as AIDS patients. Metronidazole is the standard treatment against Giardia infection, however, no suitable antimicrobial agent exists to eradicate Cryptosporidium.

Ninety percent of transmission of these pathogenic protozoans is through water while 10% occurs through food (Rose and Slifko, 1999). The incidence of foodborne outbreaks due to protozoan pathogens is likely underestimated due to the difficulty in detection of low numbers of organisms, as enrichment techniques cannot be used. Detection of Giardia and Cryptosporidium on domestic, fresh vegetables and fruits in Norway (Robertson and Gjerde, 2001), a wealthy and modern country, have important implications for food safety in North America.

Infection with these protozoans is initiated through the ingestion of the cyst stage of Giardia or oocyst stage of Cryptosporidium. These transmission stages are very hardy and can persist in the environment for a month (Giardia) or several months (Cryptosporidium). While their abundance in water is very low, from 0.5-200/100 L water with an average of 25 cysts/100 L (Wallis et al., 1994; Payment et al., 2000; Thurston-Enriques et al., 2002), the infective dose is also very low (10 cysts/oocysts; Rendtorf, 1954; DuPont et al., 1995). Thus, very sensitive techniques are required to detect cysts/oocysts in the environment. There are no standard collection methods for concentration of Giardia or Cryptosporidium from environmental samples, however, the USA EPA recommends the use of method 1623 involving filtration through Envirocheck filters and immunomagnetic bead separation (USA EPA, 1999). This procedure is very costly (>$100/sample) and filtration of water samples through envirocheck filters (Pall Gelman) is not very efficient, ranging from 15% (Simmons et al., 2001). Other methods, filtration through 3 μm cellulose nitrate and 1.2 μm cellulose acetate (Sheppard and Wyn-Jones, 1996) are much less expensive ($1/filter) and are as efficient as the Envirocheck. An alternative method has been described for simultaneous collection of protozoa, bacteria and viruses using ultra filtration membranes. The microza ultra filtration system has efficiencies of recovery of Cryptosporidium of 30-80% from environmental water samples (Kuhn and Oshima, 2001). These filters are reusable and come in different sizes to accommodate 2-1000 L volumes of water (Pall Gellman).

Cysts and oocysts are resistant to many environmental stresses and to disinfection, such as chlorination, used in water treatment practices. Distinguishing live from dead cells is important in determining water treatment effectiveness and risks to public health. Current methods for viability determination include animal infectivity (Black et al., 1996; Neumann et al., 2000), vital dye staining (Belosevic et al., 1997), excystation (Rose et al., 1988) and in vitro cultivation combined with PCR (Rochelle et. al., 1997; Rochelle et al., 2002; Di Giovanni et al., 1999). Reverse transcription PCR (RT-PCR) enables measurement of mRNA to detect viable cells and has been used to determine G. lamblia and C. parvum viability (Mahbubani et al., 1991; Stinear et. al., 1996; Jenkins et al., 2000).

Domestic animals, pets and wildlife act as reservoirs of Giardia and Cryptosporidium (Thompson, 2000; Heitman et al., 2002; Dillingham et al., 2002). A comparative study of sources of Giardia and Cryptosporidium from humans (sewage influent), agriculture (farms) and wildlife (scats) found that the lowest prevalence was in wildlife and the highest in human sewage. However, the highest concentrations of these protozoans were from calf-cow sources (Heitman et al., 2002). Prevalences of Giardia and Cryptosporidium on farms range from 9-40% in cattle, sheep, pigs and horses (Olsen et al., 1997). There is considerable genetic diversity within G. lamblia and C. parvum and both can be subdivided into major genotypes, each containing sub-genotypes. The major genotypes of G. lamblia are assemblages A and B; A is associated with a mixture of human and animal isolates and B is predominately associated with human isolates (Thompson et al., 2000). The greatest potential for zoonotic transmission of Giardia is with assemblage A genotypes. A similar pattern exists with C. parvum isolates, whereby genotype 1 contains predominately human isolates and genotype 2 contains bovine isolates (Dillingham et al., 2002). Knowledge of genotype can assist in identification of source of waterborne outbreaks for predictive epidemiology.

Methodologies for identifying pathogenic Giardia and Cryptosporidium are not nearly as well defined as for bacterial identification. They rely primarily on microscopic identification of intact cysts, requiring an expert in identification, time for staining the cells, preparing slides and examination. Stains for detection of cells include dyes such as Lugol's stain and immunofluorescent stains (e.g. Dynabeads G-C combo kit form Dynal Ltd. and Aqua-Glo G/C Direct, Waterborne Inc.). Other methods for detection of intact cysts or oocysts involve using fluorescent antibody labeling and detection by flow cytometry. Enzyme immunoassay kits are available on the market and take 2-3 hr to perform (Prospect T/Cryptos, Alexon Inc. and Giardia Celisa, CELLABS PTY LTD). Recently, a rapid antigen based kit (ColorPAC™, BD) for detection of Giardia and Cryptosporidium was recalled by the manufacturer due to false positives (MMWR, 2002). None of these techniques provide the ability to genotype.

PCR has been used to detect Giardia and Cryptosporidium in waste, ground and treated waters (Johnson et al., 1995; Stinear et al., 1996; Kaucner and Stinear, 1998; Chung et al., 1998), sewage sludge (Rimhanen-Finne et al., 2001), soil (Walker et al., 1998; Mahbubani et al., 1998), food (Laberge et al., 1996) and stool (Morgan et al., 1998; Webster et al., 1996; Gobet et al., 1997). PCR is equally or more sensitive than immunofluorescent antibody (IFA) in detection of these pathogens (Mayer and Palmer, 1996; Morgan et al., 1998) and has the capability for high throughput processing of samples resulting in significant reduction in costs.

Real-time PCR detection of Cryptosporidium has recently been reported. The primer/probe sequences have been based on: the Cp11 rRNA and 18s rRNA genes (Higgins et al., 2001); an unidentified gene segment generated by the random amplified polymorphic DNA (RAPD) technique (MacDonald et al., 2002); an oocyst wall protein encoding gene (Fontaine and Guillot, 2002); a highly polymorphic region of the SSU rRNA (Limor et al., 2002) and β-tubulin (Tanriverdi et. al., 2002). To date there have been no reports of the use of real-time PCR for detection of Giardia.

Traditional methods of bacterial detection in foods rely on cultivation of bacteria from the food matrix. While these procedures are very sensitive they can take days to produce results. Enzymatic and molecular approaches are much more rapid but the sensitivity of detection, 10³ to 10⁴ CFU/gm, is typically less than cultivation (Jaykus, 2003). Rapid techniques for concentrating and isolating bacteria from food matrixes (carcass swabs) and rapid detection of the bacteria using real-time PCR (qPCR) would greatly benefit the public by increasing the safety of their food.

From the preceding, it will be appreciated that there is an acute need for methods and reagents that enable the rapid and accurate detection of pathogenic microbes not only in environmental samples but, failing their detection and reduction, also in clinical samples of infected individuals to enable proper and rapid medical treatment. This need is especially acute with respect to total coliforms (as a water quality indicator) and such pathogenic microbes as E. coli O157:H7, the microcystin-producing cyanobacteria including M. aeuroginosa, and the protozoan parasites including Cryptosporidium such as C. parvum and Giardia including G. lamblia. It is accordingly an object of the present invention to provide methods and reagents useful in their detection.

### SUMMARY OF THE INVENTION

In one aspect, the present invention provides a method useful to detect a pathogenic microbe, the method comprising the step of subjecting a DNA sample that is either extracted from said microbe or is a cDNA equivalent to a polymerase chain reaction comprising primers adapted to produce and amplify a detectable amplicon from a gene responsible for the pathogenicity of said microbe, and measuring in real time the accumulation of said amplicon during said reaction. In a preferred embodiment of the invention, to render the amplicon detectable during the reaction, the polymerase chain reaction is performed in the presence of both an enzyme having 5′ nuclease activity (a 5′ nuclease) and a probe having a detectable label released following cleavage of the probe by the action of the 5′ nuclease.

In another aspect, the present invention provides a multiplexed method useful to detect at least two different pathogenic microbes in a given sample, the method comprising the step of subjecting a sample comprising DNA extracted from said microbes, or a cDNA equivalent thereof, to a polymerase chain reaction comprising primers adapted to produce and amplify detectable amplicons that are different for each pathogenic microbe, and measuring in real time the accumulation of said amplicons during the reaction. Desirably, the multiplexed method also utilizes the 5′ nuclease susceptible probes to detect and measure accumulation of the amplicons.

For the detection of specific pathogenic microbes, the present invention further provides oligonucleotide primers and oligonucleotide probes useful in a polymerase chain reaction to detect the presence of a selected pathogenic microbe.

In embodiments of the present invention, there is provided an amplicon having a nucleotide sequence selected from the coding region of:

(a) the region spanning residues 2574-2895 of the lacZ gene of E. coli;

(b) the region spanning residues 2673-2759 of the eaeA gene of E. coli O157:H7;

(c) the region spanning residues 1438-1559 of the mcyA gene of Microcystis aeruginosa;

(d) the region spanning residues 222-296 of the β-giardin gene of G. lamblia;

(e) the region spanning residues 411-485 of the β-giardin gene of G. lamblia; and

(f) the region spanning residues 583-733 of the COWP gene of C. parvum.

In other embodiments of the present invention, the primers and probe are adapted to detect total coliforms (tested with E. coli). In a specific embodiment, the primers are designed to produce an amplicon from the E. coli lacZ gene, which preferably is a 142 bp amplicon spanning residues 2574 and 2895 (numbered with reference to GenBank Accession: V00296). In other embodiments of the invention, there are provided primers useful in the amplification of that amplicon of the E. coli lacZ gene, which are selected from the primers identified in Table 2 herein as SEQ ID NOs: 4 and 5. In another embodiment, the present invention provides a probe useful to detect the amplicon resulting from said primers, the probe having SEQ ID NO.6. In a preferred embodiment, the probe incorporates one or more labels that are released for detection when the probe is cleaved by an enzyme having 5′ nuclease activity. With these reagents, the present method can be applied for the detection of coliforms, including E. coli strains are capable of causing intestinal disease.

In another embodiment of the invention the primers and probe are adapted to detect E. coli O157:H7. In a specific embodiment, the primers are designed to produce an amplicon from the eaeA gene, which preferably is an 87 bp amplicon located between residues 2673 and 2759 (numbered with reference to GenBank Accession: X60439). In other embodiments of the invention, there are provided primers useful in producing an amplicon of the eaeA gene, which are selected from the primers identified in Table 2 herein as SEQ ID NOs: 1 and 2. In another embodiment, the present invention provides a probe useful to detect the amplicon resulting from said primers, the probe having SEQ ID NO.3. In a preferred embodiment, the probe incorporates one or more labels released for detection when the probe is cleaved by an enzyme having 5′ nuclease activity.

In another embodiment of the invention the primers and probe are adapted to detect microcystin-producing cyanobacteria, and particularly M. aeruginosa. In a specific embodiment, the primers are designed to produce an amplicon from the mcyA gene from the microcystin synthetase gene operon, which preferably is a 122 bp amplicon spanning residues 1438 and 1559 (numbered with reference to Gen Bank Accession: AB019578). In other embodiments of the invention, there are provided primers useful in producing an amplicon of the mcyA gene, which are selected from the primers identified in Table 2 herein as SEQ ID NOs: 7 and 8. In another embodiment, the present invention provides a probe useful to detect the amplicon resulting from said primers, the probe having SEQ ID NO.9. In a preferred embodiment, the probe incorporates one or more labels that are released for detection when the probe is cleaved by the action of an enzyme having 5′ nuclease activity.

In still another embodiment of the invention, the primers and probes are adapted to detect pathogenic protozoans including Giardia and particularly G. lamblia, as well as Cryptosporidium including C. parvum. With respect to detection of G. lamblia, the primers are designed to produce an amplicon from the β-giardin gene. One set of primers, herein referred to infra as the P241 set, yields a 74 bp amplicon spanning residues 222-296 (CDS of GenBank Accession #M36728). In specific embodiments, the primers are selected from the primers identified in Table 2 herein as SEQ ID NOs: 10 and 11. In another embodiment, the present invention provides a probe useful to detect the amplicon resulting from said primers, the probe having SEQ ID NO.12. In other embodiments, the primers are designed to produce a 74 bp amplicon spanning residues 411-485 (CDS of GenBank Accession #M36728) of the β-giardin gene, and the primers, designated P434 herein, are selected from the primers identified in Table 2 by SEQ ID NOs. 13 and 14. A suitable probe for such an amplicon has the sequence represented by SEQ ID NO. 15, in Table 2 infra.

For detection particularly of C. parvum, the primers are designed to produce an amplicon from the Cryptosporidium oocyst wall protein, designated COWP. The primers suitably are designed to produce a 151 bp amplicon spanning residues 583-733 (CDS of Gen Bank Acc #Z22537). In specific embodiments, the primers are selected from the primers identified in Table 2 herein as SEQ ID NOs: 16 and 17. In another embodiment, the present invention provides a probe useful to detect the amplicon resulting from said primers, the probe having SEQ ID NO. 18.

It will be appreciated that the present invention also embraces amplicon-binding sequence variants of the primers and probes herein described. Such variants may include substitution of from 1-5 nucleotides in the noted sequences. The substitutions are selected to minimize loss in binding affinity for the amplicon that results from the substitution, relative to the actual sequences herein provided.

It will also be appreciated that the primer and probe sets herein described will be useful to produce amplicons having some variation, say up to 20% variation, from the specific amplicon sequences herein described. While some specificity may be sacrificed, the method nevertheless will still detect pathogen strains having minor variation in the sequence targeted for amplification and detection.

It is to be appreciated that while the method of the present invention preferably utilizes a real time, 5′ nuclease-based polymerase chain reaction to produce and detect the amplicon targeted within the microbial genome, the primers and probes herein described can also be used in polymerase chain reactions and related procedures that utilize different strategies, including RT-PCR, end-point PCR, NASBA and the like. In this vein, it will further be appreciated that the substrate DNA can either be extracted from the microbe(s) present in the sample, or it can be synthesized from extracted RNA using standard methods of cDNA preparation. Alternatively, the extracted RNA can serve as the intermediary of an otherwise DNA-based amplification method. In the NASBA approach, for instance, the given amplicon can be produced using the reverse primers herein described, but using a forward primer adapted by addition 5′ of 25 bp constituting the sequence for T7 promoter. In this approach, the same probe sequence can also be employed, but incorporating a molecular beacon probe instead of the Taqman probe.

It will thus be appreciated that the present invention is particularly adapted for the rapid, sensitive and selective detection, in real time, of a variety of pathogenic microbes in both environmental and clinical specimens. Embodiments of the present invention are particularly adapted for the detection of total coliforms, E. coli O157:H7, toxigenic M. aeruginosa, G. lamblia, and C. parvum.

In addition, the present invention provides improvements in procedures by which DNA samples are collected, in methodology for managing inhibitory substances in the samples, and in methods for discriminating between live and dead cells within a sample. These improvements permit analysis of a wider array of microbial samples, including finished drinking water, sewage, waste water, treated water, disinfected water, irrigation water, and water obtained from wells, rivers, lakes and recreational waters such as swimming pools. Other samples that can be analyzed by the present method include food (such as fruits, vegetables, meat and prepared food items), swabs taken from slaughter lines, and meat surfaces, as well as swabs taken from environmental surfaces from slaughter houses, and meat preparation facilities, soil and clinical and veterinary samples including stool and biopsy samples.

In the particular case of Giardia and Cryptosporidium, the present invention provides methodologies for rapid, specific and high throughput screening, using real-time PCR or other sequence-based hybridization methodologies. This enables examination of large numbers of samples to identify asymptomatic individuals shedding cysts/oocysts, providing the true prevalence of parasitaemia in communities. Additionally, simultaneous genotyping capabilities as herein provided allow for predictive epidemiology, critical for action in outbreak situations.

It will be appreciated that “real-time PCR” is distinguished from endpoint (standard) PCR in that measurements are made during DNA amplification and are done so in real-time. Standard or endpoint PCR is measured at the end of a run, is not quantitative, and may take 1 plus days to obtain results. In real-time PCR, a sequence-specific primer set and a fluorescently labeled sequence-specific probe are used for detection of a specific target. The probes utilize the 5′ exonuclease function of Taq DNA polymerase to cleave the fluorophore from the probe when bound to its target. Fluorescence is recorded over time as it accumulates with PCR cycling and it is directly proportional to the starting number of target copies in the initial sample. Real-time PCR provides accurate quantification of the target, as the target is quantified while amplification is still in the exponential part of the reaction. With multiplex real-time PCR, applied in embodiments of the present invention, the reporter dye for each target is detected simultaneously from each PCR reaction by a distinct emission wavelength (colour) after excitation by a light source. A real-time PCR diagnostics approach offers a wide concentration range in which it can detect the target organism (over 7 log units). This assay is also very sensitive, potentially detecting down to 1 copy of the target gene.

Embodiments of the present invention are now described in the examples which follow, and with reference to the accompanying drawings in which:

### BRIEF DESCRIPTION OF THE DRAWINGS

FIG. 1: Range of bacterial detection in real-time PCR as shown by amplification plots. In the multiplex plot lacZ amplification is represented by black lines and closed circles, and eae amplification is represented by grey ‘x’s. The lines represent amplification of 10-fold serial dilutions of genomic DNA.

FIG. 2: Standard curves generated from real-time PCR correspond to the amplification plots in FIG. 1. The standard curve is generated of 10-fold serial dilutions of genomic DNA standards (closed squares) from 1×10⁷ to 1×10⁰ copies of eaeA/μl and 2×10⁷ to 2×10⁰ copies of lacZ/μl and shows sample starting concentration (open squares).

FIG. 3: Range of protozoan detection in real-time PCR as shown by amplification plots. G. lamblia was detected using the β-giardin P241 primer/probe set and C. parvum by the COWP gene. The β-giardin and COWP plots demonstrate 10-fold serial dilutions and 2-fold serial dilutions were used to generate the multiplex amplification plot.

FIG. 4: Standard curves generated from real-time PCR correspond to the amplification plots in FIG. 3. In panel 1 (β-giardin) 10 fold serial dilutions ranging from 25 ng to 25 fg of DNA corresponds to 1.3×10⁵ to 1 cyst. The standard curve for the COWP gene represents 10 fold serial dilutions of C. parvum DNA, from 5.7 ng to 5.7 fg and correspond to 1×10⁵ to 1 oocyst. The multiplex standard curves were generated from 2 fold dilutions of DNA ranging from 2.5 ng to 390 fg.

### DETAILED DESCRIPTION OF THE INVENTION

### EXAMPLES

Detailed descriptions of the methods used for detecting these organisms using real-time PCR are provided in the following examples. Differences in size and abundance in environmental samples between the 4 pathogens described herein necessitated the development and utilization of a variety of methods for collection and concentration of the pathogens from samples. For example, bacteria were enumerated on 100 ml water samples using a 0.2 um pore size filters due to their small size whereas, 2 L water samples were concentrated for detection of protozoa and 1 to 3 um pore size filters employed. Similarly, the variation in hardiness of the cell wall of these organisms necessitated the use of different DNA extraction methods for efficient DNA extraction.

### Example 1

Bacterial Strains and Culture Conditions

The bacterial strains and isolates of protozoans used and the culture conditions are listed below.

E. coli (ATCC 8739) were cultured nutrient broth and incubated at 37° C., overnight on a rotary shaker (New Brunswick Scientific Co.) at 200 rpm, or mainteined on nutrient agar (2%) plates. Cell population densities were quantified spectrophotometer (DU-64; Beckman) at 550 nm.

E. coli O157:H7 (ATCC 35150, Oxoid Inc.) were maintained on tryptic soy agar E. coli O157:H7 was cultured overnight at 37° C. on a shaker in tryptic soy broth (TBS) and for selective identification on Sorbitol MacConkey Agar containing cafeximine and telliurite (CT-SMAC; Oxoid) at 37° C. for 24 hours.

M. aeruginosa cultures (UTCC 300, 468, and 459) were maintained in liquid BG-11 medium (Rippka et al., 1979) at 25° C. on a shaker (150 rpm) under a flourecent light source 25-30 μEinm⁻² s⁻¹. Strains were subcultured every two weeks. Cell population densities were quantified with a spectrophotometer (DU-64; Beckman) at 730 nm.

Protoza:

Giardia cysts: Live G. lamblia cysts, produced by passage of the human strain CH3 of G. intestinalis through Mongolian gerbils, were purchased from Waterborne Inc. (New Orleans, La.). Cysts were delivered in PBS containing antibiotics, stored at 4° C. used within 1 month. The WB strain was obtained Dept. Biology, University of Alberta. The GA strain was obtained by extraction of DNA from cysts obtained from fecal sample of a patient in Ontario, Canada. G. muris Roberts-Thompson strain obtained from Waterborne Inc.

Cryptosporidium oocysts: Live C. parvum oocysts (IOWA strain) produced by passage in calves were purchased from Waterborne Inc., delivered in PBS containing antibiotics, stored at 4° C. and used within 3 months. Live oocysts of the GCH1 isolate were obtained through the NIH AIDS Research and Reference Reagent Program, Division of AIDS, NIAID, NIH: contributed by Dr. Saul Tzipori.

### Example 2

Collection and Concentration from Water Samples

The methodologies for optimal collection and concentration of E. coli, M. aeruginosa, G. lamblia, and C. parvum are organism dependent.

E. coli (and coliforms) and Microcystis:

Collection from Water: Water samples were examined for the presence of E. coli and Microcystis. Environmental samples were collected in wide mouth 500 ml polypropylene bottles (VWR, Mississauga, ON). Collected environmental (100 ml) and bottled water (100-500 ml) samples were concentrated onto 0.2 μm membranes (47 mm Supor™, Pall Gelman, Mississauga, ON) by vacuum filtration in Nalgene® filter units with receivers (model 300-4000; VWR, Mississauga, ON). In each experiment filtered MQ water was processed as a negative control and bacterially spiked water samples were processed as positive controls.

Collection and concentration of bacteria from Sponges: Sponges were placed into sterile bags and 50 ml of ddH₂O containing 0.2% of Tween 20 was added to each bag. The bags were pulsified for 15 sec in a Pulsifier (Microbiology International). The homogenates were concentrated onto 0.2 μm membranes (47 mm Supor™, Pall Gelman, Mississauga, ON) by vacuum filtration in Nalgene® filter units with receivers (model 300-4000; VWR, Mississauga, ON). The sponges in the bag were washed two times using 50 ml of ddH20 by rigorous shaking and each wash was concentrated onto the filters. DNA was extracted from the filters using the procedure described in example 3.

Collection and concentration of bacteria from sponge swabs after growth in enrichment media: Sponges inoculated with E. coli were placed in 125 ml of nutrient broth or Tryptic soy broth (TSB) in wide mouth 500 ml polypropylene bottles (VWR, Mississauga, ON) and were left on a shaker for 2 to 5 hr, at 37° C. Enriched media samples (25-35 ml) were concentrated onto 0.2 μm membranes (47 mm Supor™, Pall Gelman, Mississauga, ON) by vacuum filtration in Nalgene® filter units with receivers (model 300-4000; VWR, Mississauga, ON). Tween 20 (0.25%) was added to the culture media before collecting on the supor membranes. For each 35 ml of media concentrated on the filter, the filter was washed with 25 ml of 25% ETOH followed by 100 ml of water. In each experiment filtered MQ water was processed as a negative control and bacterially spiked water samples were processed as positive controls. DNA was extracted from the filters using the procedure described in example 3.

Giardia and Cryptosporidium:

Vacuum Filtration: Water samples were collected in 10 L plastic carboys (Cole Palmer, Chicago, Ill.) and stored at 4° C. until use (same day). Samples (2 L) were filtered through 3 μm cellulose nitrate filters, 47 mm diameter (Sartorius, Goettingen, Germany) in a parabolic stainless funnel (Gelman, Ann Arbor, Mich.) using a vacuum pressure between 10-15 PSI generated by a Millipore Vacuum/Pressure pump (115V,60 Hz; Millipore,). Following filtration of the sample, the funnel was rinsed with double-distilled (dd) water. Cellulose acetate filters, with a pore size of 1.2 μm were used for collection of C. parvum by vacuum filtration. For simultaneous detection of Giardia and Cryptosporidium from a single sample the sample was filtered through a 3 μm cellulose nitrate filter (as described above) and the filtrate was filtered through a 1.2 μm cellulose acetate filter.

### Example 3

DNA Extraction

To evaluate the efficiency of DNA extraction for E. coli, M aeruginosa, G. lamblia, and C. parvum different extraction procedures were evaluated for the different organisms and different types of samples. The commonly adopted methods are described below.

E. coli (and coliforms): DNA extraction membranes from the collection units, described above in example 2 was aseptically transferred into a 2 ml screw-cap microfuge tube and 200 μl of PrepMan™Ultra (ABI, Foster City, Calif.) was added and the tube was vortexed to disperse the sample. The sample was then heated to 100° C. in a water bath for 10 min. The samples were removed and allowed to cool for 2 min, then briefly centrifuged to transfer the supernatant to a clean microfuge tube. This one step procedure allows use of the extract directly in the 5′ nuclease real-time PCR reactions.

Microcystis: DNA extraction membranes were aseptically transferred to a 1.5 ml microfuge tube from the filtration units. The DNeasy Tissue kit (Qiagen, Mississauga, ON) was used for DNA extraction from the cells on the membrane, using a modified method DNA extraction from Gram negative bacteria. The membrane was suspended in 360 μl ATL buffer and 40 μl Proteinase K, vortexed and incubated at 55° C. for 1hr to overnight. The sample was vortexed for 15 sec, and 400 μl of AL buffer was added. The sample was vortexed again and incubated at 70° C. for 10 min, 400 μl of absolute ethanol was added the sample was vortexed again. The manufacturer's protocol was followed onward and DNA was eluted in two steps with 50 μl AE buffer.

Giardia and Cryptosporidium:

DNeasy Kit: DNA was extracted from cysts/oocysts using the DNeasy Tissue kit (Qiagen, Hilden, Germany). A modification of the animal tissue protocol was employed: 1). Tubes containing the pellet of cysts or oocysts were taped to dislodge the cells, suspended in 180 μl ATL plus 20 μl of Proteinase K and incubated for 1 hr in a 56° C. water bath; 2) cells were subjected to 3 cycles of freeze/thaw, each cycle consisting of 2 min each in liquid nitrogen followed by boiling water; 3). 3 bursts of sonication, each of 20 sec duration, using a microprobe on a Model W-220F Cell Disruptor (ULTRASONICS INC) or alternatively, 30 min sonication in a 2½″ cup horn (Sonics and Materials Inc., Newtown, Conn.), or 2 min vortex in the presence of 0.02 gm of 425-600 μm glass beads (Sigma, St. Louis, Mo.). DNA was quantified using the PicoGreen® dsDNA quantitation reagent (Molecular Probes, Seattle, Wash.). The manufacturer's protocol volumes were reduced to obtain a 50 μl total reaction volume and 10 μl of sample was added to each well. Fluorescence was determined using the FAM filter set in an Mx4000 (Stratagene). The use of the DNeasy kit with freeze/thaw and sonication yielded 100% efficient extraction of DNA based on comparison of DNA concentration measured by PicoGreen, compared with the theoretical yield of DNA/cyst or oocyst.

Extraction of DNA from filters following concentration of environmental water samples: Cellulose nitrate and cellulose acetate filters were removed, folded twice, lengthwise with the upper surface facing out and placed into Eppendorf tubes. DNA was extracted directly from the filter using the DNeasy kit (Qiagen). Following incubation in 180 μl ATL and 20 μl proteinase K for 1 hr at 56° C. the filter was washed with 200 μl of ATL and the wash pooled with the initial cell lysate. The procedure outlined in example above was followed to extract DNA from the cells. DNA was eluted from the column using either 1 round of 50 μl dd water or 2 rounds of 50 μl dd water.

Extraction of Giardia DNA from stool: DNA was extracted from stool using the QIAamp® DNA stool kit (Qiagen) with modifications. An aliquot of 0.2 gm of SAF-fixed stool was washed twice in sterile phosphate-buffered saline, pH 7.2 (PBS), by centrifugation at 12,000×g for 10 min. The supernatant was removed and the pellet was suspended in 0.6 ml of ATL buffer (Qiagen, Germany) and incubated in a 56° C. water bath for 4 hr. The sample was subjected to 3 cycles of freeze/thaw (as described above) and incubated at 56° C. overnight. After three, 20 sec bursts of sonication, an additional 0.6 ml ATL was added to each tube, the contents mixed by vortex for 15 sec and split equally into two tubes. Half an inhibitex tablet was added to each tube containing sample and the manufacturer's procedure for the QIAamp® DNA stool kit (Qiagen) was followed. DNA was eluted from the silica gel column using 2 rounds of 100 μl sterile, dd water. Samples were stored at −20° C. until use.

Extraction of Giardia and Cryptosoridium from raw sewage: One L raw sewage samples were centrifuged at 3,000×g for 30 min to pellet cells. DNA was extracted directly from the pellet by the following method. The pellets were resuspended in ATL lysis buffer and proteinase K and inhibitor removers were added to the sample: Chelex® (BIO RAD) slurry, to a final concentration of 20% and PVP-360 (ICN, Aurora, Ohio), to a final concentration of 2%. The samples were incubated for 30 min at 56° C., subjected to freeze/thaw and sonication and centrifuged at 12,000×g for 10 min. The supernatant was processed on two DNeasy columns following the manufacturer's description and eluted from the column using 2 volumes of 50 μl of dd water. The samples were pooled to equal a total volume of 200 μl.

### Example 4

Oligonucleotide Design

Upon selection of a gene of interest to serve as a target for 5′ nuclease PCR, subsets of the target gene were selected as regions for oligonucleotide design based on regions of low homology to other targets from a blastn search (NCBI). From subsets of blastn hits, regions that showed high homology to other microorganisms, especially those likely to be found in water, food, or clinical samples were excluded. The gene domains with the lowest levels of homology were used in Primer Express Software (ABI) that generated an output list of 200 possible primer/probe combinations the list was refined and regenerated for a specific oligonucleotide within a set until the desired parameters were met. From the generated oligonucleotide combinations, selections were based on % GC content, GC relative distribution, strings of identical nucleotides, secondary structure, and Tm. All selected oligonucleotides were subjected to a blastn analysis on GenBank (NCBI) prior to synthesis, to ensure specificity for detection of the target organism. Primers and probes were synthesized using standard methodology. The probes were 5′ labeled with either FAM (6-carboxyfluroescein, λₑₘ=518 nm), HEX (5′-Hexachloro-Fluorescein, λₑₘ=553 nm), JOE (6-carboxy-4′,5′-dichloro-2′,7′-dimethoxyfluorescein, λₑₘ=548 nm) or Cy5 (1-(epsilon-carboxypentyl)-1′-ethyl-3,3,3′,3′-tetramethylindodicarbocyanine-5, λₑₘ=667 nm); both probes were also 3′ labeled with a non-fluorescent Black Hole Quencher (BHQ) dye (Biosearch Technologies Inc.; IDT Technologies).

E. coli (and coliforms): A lacZ (GenBank Acc #V00296) primer and probe set was designed to detect the beta-galactosidase gene, and recognizes both total coliforms (including non-toxigenic E. coli and the toxigenic strain, E. coli O157:H7. A general indicator that would encompass coliform bacteria is lacZ, encoding the enzyme β-D-galactosidase, which is present in all coliforms (Apte et al., 1995), including E. coli O157:H7.

E. coli O157:H7: We have also designed an eaeA primer set and probe to detect the 3′ end of the attaching and effacing gene, encoding intimin, (GenBank Acc #X60439) of E. coli O157:H7.

Microcystis aeruginosa: To distinguish between toxic-microcystin producing cyanobacteria and non-toxic forms, the MISY primer set was designed to amplify a region of the mcyA (GenBank Acc #AB019578) gene from the microcystin synthetase gene operon, involved in the synthesis of the microcystin toxin (122 bp amplicon). McyA is directly involved in biosynthesis of the toxin, and disruption mutants do not produce detectable levels of microcystins (Tillett et al., 2000). McyA is part of the peptide synthetase module of the microcystin synthetase gene operon, ins ertional mutagenesis into this gene abolished toxin production (Nishizawa et al., 2000).

These mcyA primers were found to be specific to toxic strains of M. aeruginosa and did not yield any amplification products from any of the other cyanobacterial or eubacterial species examined (M. aeruginosa (strains UTCC 300, UTCC 459, UTCC 468, and PCC7005), A. flos-aquae (strains AF67 and AF64); non-toxigenic E. coli (ECUTM), Bacillus subtilis (UTM 206), Proteus vulgaris (BCC 219), and Enterobacter aerogenes (BCC 208)). The 5′ nuclease PCR results discriminated between toxic strains of M. aeruginosa (MA459, MA300) and a non-toxic strain (MA468). There was no increase in fluorescence detection above background for non-toxic MA468 samples in real-time PCR experiments (Ct of 40).

G. lamblia: Two primer/probe sets were designed against the complete coding sequence of the β-giardin gene (GenBank Accession #M36728) of the Portland-1 strain of G. lamblia (Holberton et al., 1995). This gene codes for a structural protein that is a component of the adhesive disk of the parasite, important in binding of trophozoites to the intestinal epithelium of their host. Two distinct primer/probe sets were designed, the first primer set P241 was based on the region 222-296 and the second set, P434, was based on region 411-485 of β-giardin (GenBank Accession #M36728) (Table 2).

C. parvum: The Cryptosporidium oocyst wall protein (COWP) (GenBank Accession #Z22537) was selected as the target gene for designing the primer probe set for detection of C. parvum. This gene was selected because it codes for a protein that is important in maintaining the integrity of the oocyst wall allowing the parasite to withstand harsh environmental factors until ingested by a new host. In designing the sequences, 26 partial sequences coding for the oocyst wall protein, from different isolates and species of Cryptosporidium were examined to identify regions of the gene specific to C. parvum and to specific genotypes 1 and 2 of C. parvum. These sequences were entered into the BIMAS www READSEQ Sequence Conversion program for conversion into a format readable by ClustalW. The converted sequences were entered into the ClustalW program (European Bioinformatics Institute) and a multiple alignment performed to identify regions of the gene. The sequences and their GenBank Accession #'s are as follows: C. parvum CBAHI (#AJ310765), C. baleyi (#AF266276), C.spp715-dog (#AF266274), C. felis (#AF266263), C. spp815-bullsnake (#AF266277), C. meleagridis (#AF248742), C. meleagridis (AF266266), C. wrairi (#AF266271), C. wrairi (U35027), C. parvum G2 (#AF248743), C. parvum CPACH-1 (#AJ310766), C. spp6-bovine (#AF266273), C. parvum G2 (#AF161577), C. spp 4A-mouse (#AF266268), C. spp-monkey (#AF266272), C. parvum G1 (#AF248741), C. parvum 181 (#AF266265), C. parvum G1 (#AF161578), C. spp 351-ferret (#AF266267), C. spp 428-kangaroo (#AF266269), C. spp 499-pig (#AF266270), C. serpentis (#AF266275), C. serpentis (#AF161580), C. andersoni (#AAF266262), C. muris (#AF266264) and C. muris (#AF161579).

The region selected for C. parvum detection ranged from 583-733 of the coding sequence of the COWP gene (GenBank Acc. #Z22537).

TABLE 1
Primer and Probe sequences.

| | | | SEQ | Location within | Amplicon |
|---|---|---|---|---|---|
| Target | Oligo^{‡} | Sequence (5′ to 3′) | ID | gene (CDS) | Size (bp) |
| eaeA | F | aataactgcttggattaaacagacatct | 1 | 2673-2700 | 87 |
| | R | ggaagagggttttgtgttattaggtt | 2 | 2734-2759 | |
| | P | aagtgcttgatactccagaacgctgctca | 3 | 2703-2731 | |
| lacZ | F | ggatctgccattgtcagacatg | 4 | 2754-2775 | 142 |
| | R | ctgttgactgtagcggctgatg | 5 | 2874-2895 | |
| | P | taccccgtacgtcttcccgagcg | 6 | 2778-2800 | |
| mcyA | F | cgaccgaggaatttcaagct | 7 | 1438-1457 | 122 |
| | R | agtatccgaccaagttacccaaac | 8 | 1536-1559 | |
| | P | ttaaatcggaaattatcccagaaaatgccgt | 9 | 1459-1489 | |
| β- | F | catccgcgaggaggtcaa | 10 | 222-239 | 74 |
| giardin | R | gcagccatggtgtcgatct | 11 | 296-278 | |
| P241 | P | aagtccgccgacaacatgtacctaacga | 12 | 241-268 | |
| β- | F | cctcaagagcctgaacgatctc | 13 | 411-432 | 74 |
| giardin | R | agctggtcgtacatcttcttcctt | 14 | 485-462 | |
| P434 | P | ttctccgtggcaatgcccgtct | 15 | 434-455 | |
| COWP | F | caaattgataccgtttgtccttctg | 16 | 583-607 | 150 |
| | R | ggcatgtcgattctaattcagct | 17 | 733-711 | |
| | P | tgccatacattgttgtcctgacaaattgaat | 18 | 702-672 | |

^{‡}Forward (F) and reverse (R) primers and dual-labeled hydrolysis probe (P); the probe for eaeA was 5′ labeled with JOE, the lacZ probe was 5′ labeled with FAM, the β-giardin probes P241 and P434 were FAM labeled and the COWP probe was labeled with HEX. Probes were 3′ quenched with TAMRA or BHQ-1 (Biosearch Technologies, Inc.).
CDS = coding sequence.

### Example 5

Real-Time PCR Conditions

Real-time (5′ nuclease) PCR reactions were carried out using reagents from the Brilliant™ qPCR kit (Stratagene, La Jolla, Calif.). Each reaction contained 4 mM MgCl₂, 800 nM dNTPs, 8% glycerol, 0-100 μg/ml BSA, 20 nM ROX (6-carboxy-X-rohdamine) normalizing dye, 1.25 U SureStart Taq DNA polymerase, 200 nM probe, 300-900 nM (Table 3) of each primer, and 1-10 μl template in a 25 μl reaction. Alternatively, for samples known to contain a low concentration of target DNA, reaction volumes were increased to 50 or 100 μl to allow addition of larger volumes of template. Reactions were carried out in an Mx4000 (Stratagene), with a 10 min incubation at 95° C., followed by 40 cycles of 15 sec at 95° C. and 1 min at 60° C. Three fluorescence readings were collected at the end of each 60° C. cycle. Each sample was run in triplicate and data analyzed using the Mx4000 software (Stratagene). Similar results were obtained when the reactions were performed in an SDS 7700 (ABI).

TABLE 2
Final concentration of oligonucleotides in real-time PCR reactions

| | | Working |
|---|---|---|
| Target | Oligo^{‡} | Concentration (nM) |
| eaeA | F | 900 |
| | R | 900 |
| | P | 200 |
| lacZ | F | 300 |
| | R | 300 |
| | P | 200 |
| mcyA | F | 50 |
| | R | 300 |
| | P | 200 |
| β-giardin P241 | F | 600 |
| | R | 300 |
| | P | 200 |
| β-giardin P434 | F | 300 |
| | R | 300 |
| | P | 200 |
| COWP | F | 300 |
| | R | 300 |
| | P | 200 |

Elimination of E. coli DNA contamination of Taq reagent:

Currently, commercial Taq polymerases are produced as recombinant proteins in E. coli and contain low levels of E. coli DNA (≦1 pg of DNA, personal communication Stratagene). When used in qPCR detection of the LacZ gene of E. coli the negative controls produce Ct values due to the bacterial DNA contamination of certain lots of the Taq reagent. These numbers mask the qPCR detection of 1,000 or fewer E. coli in the samples. For this reason contaminating DNA will be destroyed using restriction enzyme digestion.

To remove DNA contamination from the Taq polymerase, the polymerase was subjected to Mbo II digestion. There is one Mbo II cutting site in the middle of the LacZ probe sequence. An aliquot of 1 ul containing 5 Units of Mbo II was added to the qPCR master mix containing the 10×buffer, water, dNTPs and Taq polymerase. The sample was incubated for 15 min at 37° C. followed by inactivation of Mbo II at 95° C. for 5 min. Once cooled, the primers, probe, reference dye and glycerol were added to the master mix and the qPCR assay was performed.

Mbo II treatment removed the Ct values in negative controls for LacZ detection (Table H). Temperature treatment of the master mix did not alter the detection compared with no treatment (not shown). There was a 1-log reduction in detection of spiked DNA (5×10⁴ copies to 5×10¹ copies) following Mbo II treatment (Table H). No Ct values were observed in the negative controls when detecting the eaeA gene for the toxigenic E. coli O157:H7 in the qPCR assay. There is one Mbo II restriction site in the reverse primer region of the eaeA amplicon. Digestion of Taq polymerase using Mbo II and inactivation of the enzyme prior to the qPCR assay did not significantly alter detection of the eaeA target.

Restriction digestion of Taq polymerase using Mbo II will be used whenever commercial lots of Taq polymerase contain DNA that is measurable in the qPCR assay for detection of the LacZ gene of coliforms.

TABLE 3
Mbo II Treatment of Taq Polymerase for qPCR
Detection of LacZ and eaeA.

| | Cycle Threshold (Ct) | | | |
|---|---|---|---|---|
| | LacZ | | eaeA | |
| PCR Template | No Mbo II | Mbo II | No Mbo II | Mbo II |
| ddH₂0 | 38.47 ± 0.94 | No Ct | No Ct | No Ct |
| −ve Filter* | 34.78 ± 0.53 | No Ct | No Ct | No Ct |
| 5 × 10⁴ copies | 22.41 ± 0.43 | 28.78 ± 0.56 | 20.60 ± 0.33 | 21.61 ± 0.25 |
| 5 × 10³ copies | 27.07 ± 0.71 | 34.59 ± 0.67 | 24.50 ± 0.33 | 25.57 ± 0.36 |
| 5 × 10² copies | 31.86 ± 0.19 | 38.70 ± 1.49 | 28.69 ± 0.02 | 29.32 ± 0.31 |
| 5 × 10¹ copies | 36.05 ± 0.26 | No Ct | 31.79 ± 1.15 | 32.18 ± 0.54 |

*−ve Filter, extraction of a filter treated with water only.

The ddH20 and -ve Filter templates were used as negative controls.

Copies of E. coli and E. coli O157:H7 DNA for detection of the LacZ and eaeA, respectively.

### Example 6

Sensitivity and Specificity of Real-Time Primer/Probe Oligonucleotides

E. coli

Microcystis aeruginosa:

Giardia and Cryptosporidium:

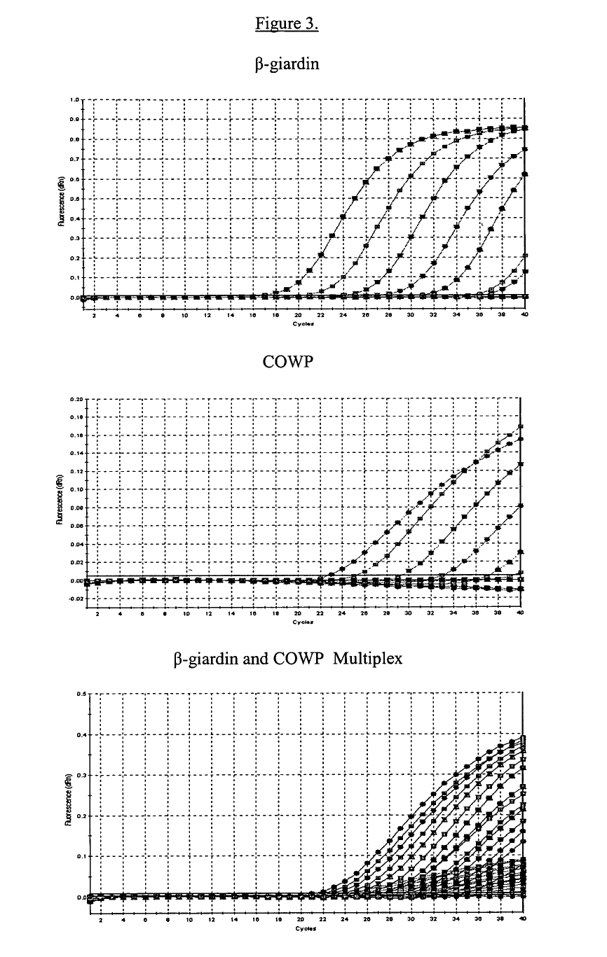

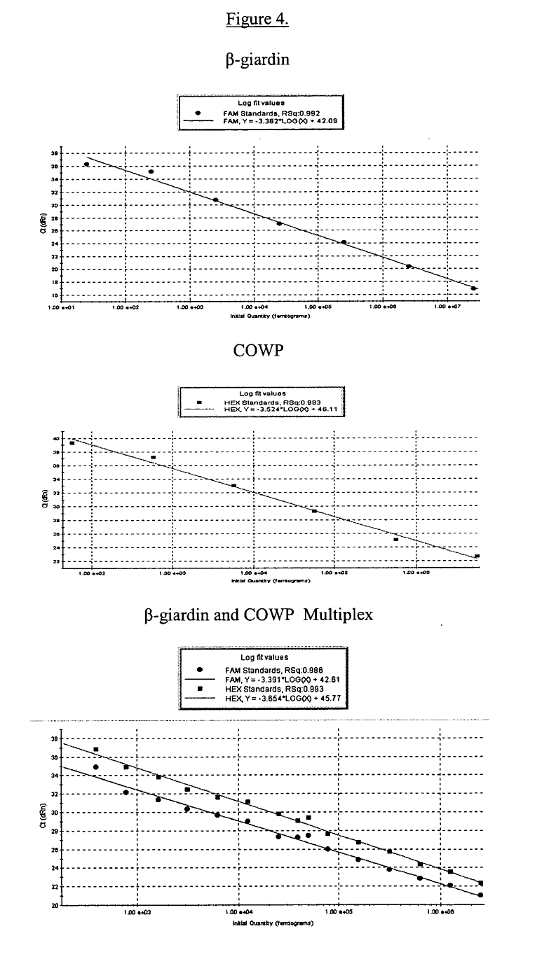

The β-giardin P241 and P434 primer/probe sets were very sensitive in detecting DNA extracted from Giardia cysts and detected DNA across a broad range of dilutions 7 logs, from as few as 1 cyst to as many as 5×10⁵ (FIGS. 3 and 4). Detection of C. parvum oocysts was in the same range, with the capability of detecting 2 oocysts. Detection of higher concentrations of Giardia and Cryptosporidium is possible when using larger starting number of cells in the DNA extraction. The primer/probe sets did not detect other unrelated sources of DNA (eg. E. coli, O. novo ulmi) in real-time PCR demonstrating specificity to the organisms they were designed to detect (Table 4). Probe 241 detects both G. lamblia and G. muris whereas P434 detected G. lamblia only.

TABLE 4
Specificity test of Oligonucleotides by Endpoint or Real-time PCR

| | | E. coli | M. | G. lamblia | G. lamblia | C. |
|---|---|---|---|---|---|---|
| DNA Source^{a} | E. coli^{b} | O157:H7^{b} | aeruginosa^{b} | P241^{c} | P434^{c} | parvum^{c} |
| A. flos-aquae | − | − | − | nd | nd | nd |
| (AF64) | | | | | | |
| A. flos-aquae | − | − | − | nd | nd | nd |
| (AF67) | | | | | | |
| B. cereus | − | − | − | nd | nd | nd |
| B. subtilis | − | − | − | nd | nd | nd |
| C. parvum | nd | nd | nd | − | − | + |
| E. aerogenes | nd | nd | − | nd | nd | nd |
| E. coli (ATCC | + | − | − | − | − | − |
| 8739) | | | | | | |
| E. coli O157:H7 | + | + | nd | nd | nd | nd |
| G. lamblia H3 | nd | nd | nd | + | + | − |
| G. lamblia WB | nd | nd | nd | + | + | − |
| G. muris | nd | nd | nd | + | − | − |
| M. aeruginosa | − | − | +^{b,c} | − | − | − |
| (UTCC 300) | | | | | | |
| M. aeruginosa | − | − | −^{b,c} | nd | nd | nd |
| (UTCC 468) | | | | | | |
| M. aeruginosa | − | − | +^{b,c} | nd | nd | nd |
| (UTCC 459) | | | | | | |
| M. aeruginosa | − | − | − | nd | nd | nd |
| (PCC 7005) | | | | | | |
| M. aeruginosa | − | − | nd | nd | nd | nd |
| (PCC 7806) | | | | | | |
| O.novo-ulmi | nd | nd | nd | − | − | − |
| (VA30) | | | | | | |
| P. vulgaris | − | − | − | nd | nd | nd |
| nd = not determined | | | | | | |
| ^{a}DNA from Anabena flos-aquae (AF 64 and AF 67), Bacillus cereus, Bacillus subtilis, Enterobacter aerogenes (Brock Culture Collection, BCC 208), Esherichia coli (ATCC 8739), Enterobacter aerogenes (Brock Culture Collection, BCC 208), Esherichia coli O157:H7 (ATCC 35150), Giardia | | | | | | |
| # lamblia (H3 and WB), Giardia muris (Roberts-Thompson strain), Microcystis aeruginosa(strains UTCC 300, UTCC 459, UTCC 468, Pasteur Culture Collection (PCC 7005 and PCC7806), Ophiostoma novo-ulmi, and Proteus vulgaris (BCC 219) | | | | | | |

^{b}Specificity of primers as detected by amplification of specific fragment in endpoint PCR.
^{c}Specificity of primers and probe as detected by emission of fluorescence in real-time PCR.

### Example 7

Standard Curves for Quantitation of Pathogenic Organisms

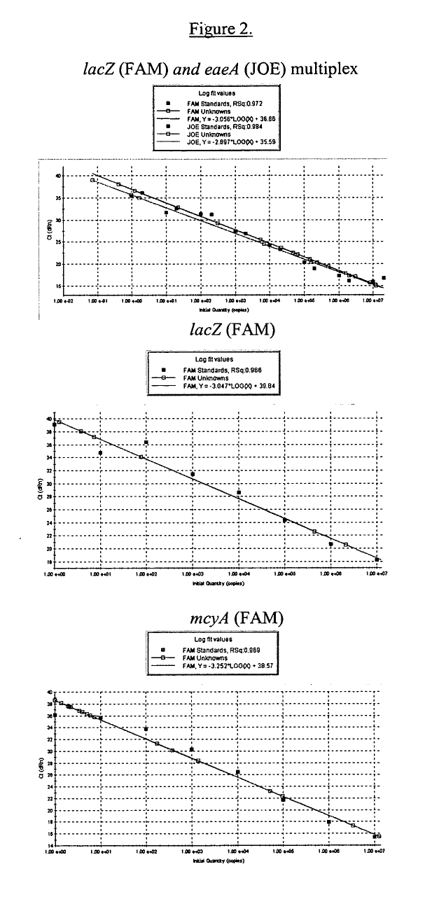

To enable quantitation of cells per sample, standard curves were generated for all 4 target organisms (FIGS. 2 and 4).

E. coli

Cell cultures were divided into 1 to 1.5 ml aliquots for DNA extraction with the DNeasy Tissue Kit (Qiagen). The manufacturer's protocol for extraction from Gram negative bacteria was followed, and elution was performed with 20 mM Tris-HCl in two steps of 25 to 50 μl each. The DNA was serially diluted and used to generate the standard curve (see example 5, real-time PCR).

Standard curves were constructed from E. coli genomic DNA of a known concentration, as determined spectrophotometrically (OD₂₆₀). The gene copy number, for lacZ or eaeA, was calculated based on the genome sizes of E. coli (4.6 Mb) and E. coli O157:H7 (5.5 Mb), respectively (GenBank); with lacZ and eaeA as single copy genes. The calculation was based on the following equation:

[DNA, g/ml]×6.0221367×10²³ gene copies/mol genome size, bp×2 b/bp×330 g/mol/b

where b=base, and bp=base pair. Standards ranged from 1×10⁷ gene copies/μl (5 μl of template were added each 25 μl reaction) to 1×10⁰ copies/μl, as obtained by 10-fold serial dilutions. DNA was also extracted (as above) from samples spiked with different relative concentrations of each bacterial strain (unknowns), to obtain quantitative results on the starting concentration of each type of E. coli in the unknown samples. Each sample was run in triplicate and a no template control was used in each PCR run.

Protozoa:

Standard curves were generated using serial dilutions (10, 5 and 2 fold dilutions) of DNA purified from cysts/oocysts, using the maximum efficiency (100%) method of extraction (DNeasy with freeze/thaw and sonication) and Picogreen dsDNA quantitation. Both the β-giardin and COWP genes are expressed as single copy genes within the nuclei. Cysts of Giardia contain 2 trophozoites that have undergone multiple steps of nuclear division and thus 16 copies of total genetic information are contained within each cyst (Bernander et al., 2001). Within Cryptosporidium oocysts are 4 nucleated sporozoites. Therefore, there are 16 copies of the β-giardin gene available in each Giardia cyst and 4 copies of the COWP gene per oocyst. The total genome sizes are 12 MB and 10.4 MB, for Giardia and Cryptosporidium, respectively.

Using the conversion: Mass (pg)=bp/0.9869×10⁹. The DNA mass of Giardia is 0.195 pg/cyst and is 0.04 pg/oocyst for Cryptosporidium.

### Example 8

Multiplex Assays

Multiplex assays for detection of 2 or more organisms in one sample significantly reduce the labour and supply costs when performing large numbers of samples. Described herein are 2 multiplex assays using sequence-specific primer/probe sets.

E. coli

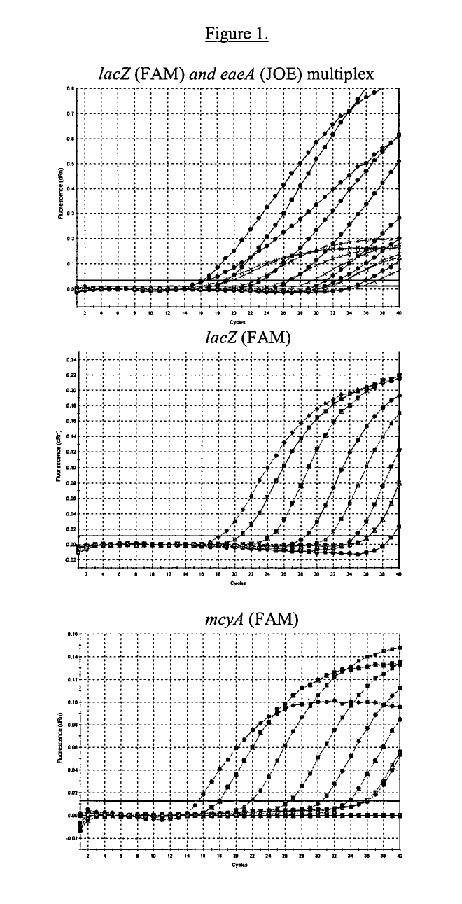

The probes for the lacZ and eaeA gene targets have been labeled with different fluorogenic probes (FAM and JOE, respectively), and can successfully identify both the toxigenic and non-toxigenic forms of E. coli in the same reaction run (FIGS. 1 and 2).

G. lamblia and C. parvum

A multiplex real-time PCR assay using β-giardin (FAM-labeled) and COWP (Hex-labeled) detected G. lamblia and C. parvum with equivalent sensitivities to a singleplex assay (see amplification plots and standard curves, FIGS. 3 & 4). Additionally, the amplicons generated by multiplex PCR were sequenced and proved to be identical to amplicons generated in the singleplex PCR.

### Example 9

Real World Application of Real-Time PCR to Detection of E. coli in Water

We have applied real-time PCR to the detection of E. coli in lake water (Table 5) and bottled drinking water (Table 6).

TABLE 5
Comparison of total E. coli cells/100 ml measurements
from Heart and Professor's Lake in Peel Region, Ontario,
obtained by culturing versus with 5′ nuclease
PCR on Jul. 31, 2002.

| | MOH Plate Count^{a} | UTM 5′ Nuclease PCR^{b} |
|---|---|---|
| Site | (cells/100 ml) | (cells/100 ml) |
| 1A | 220 | 165 |
| 2A | 20 | 73 |
| 3A | 20 | 111 |
| 4A | 50 | 187 |
| 5A | 20 | 72 |
| 6A | 50 | 50 |
| 1B | 60 | 128 |
| 2B | 10 | 43 |
| 3B | 10 | 128 |
| 4B | 20 | 77 |

^{a}Counts obtained from the Ontario Ministry of Health and Long Term Care (MOH) by culturing 10 ml of water from Heart and Professor's Lakes on media. Counts were rounded up to the nearest 10 cells/100 ml.
^{b}Counts obtained by performing multiplex 5′ nuclease PCR (to detect total E. coli and toxigenic E. coli O157: H7, by amplification of lacZ and eaeA, respectively), by concentrating 100 ml of water from Heart and Professor's Lakes and extracting DNA prior to performing 5′ nuclease PCR.

TABLE 6
Colony Growth and Endpoint and Real-Time PCR
Quantification of total E. coli in Commercially
Sold Bottled Water

| | | | % Bottles with | |
|---|---|---|---|---|
| | | % Bottles with | LacZ | |
| Bottled | | LacZ | Amplification | Real-time PCR |
| Water | % Colony^{a} | Amplification with | with Real-Time | Concentration Range |
| Brand | Growth | Endpoint PCR^{b} | PCR (fraction) | (copies or cells/bottle) |
| E | 44 | 22 | 64 | 14.00 ± 2.47-2.50 ± 0.00 |
| | | | (7/11) | |
| F | 33 | 44 | 56 | 6.00 ± 0.85-2.50 ± 2.02 |
| | | | (5/9) | |
| G | 33 | 11 | 17 | 7.00 ± 11.2-3.00 ± 1.89 |
| | | | (2/12) | |
| H | 11 | 22 | 33 | 4.00 ± 0.61-3.00 ± 1.09 |
| | | | (4/12) | |

^{a}Colony growth on LB solid media, with incubation for 24 hr at 37° C.
^{b}21 bottles per brand were sampled from three different lot numbers

### Example 10

Protozoan Genotype Determination

Primer and probe set P241 amplifies and detects all the strains of G. lamblia and the G. muris spp, whereas primer and probe set P434 is dependent on the sequence of the strain. Sequence variation within this region of the β-giardin gene (411-485) provides a means of genotyping G. lamblia. Oligonucleotides based on the coding sequence of the β-giardin gene of the Portland-1 strain of G. lamblia (GenBank Acc. #M36728) detect assemblage A isolates and oligonucleotides based on the H3 isolate sequence (sequenced in our lab) detect assemblage B (Table 7). These are specific to G. lamblia assemblages and do not detect G. muris, the murine species of Giardia (Table 8).

Use of molecular beacon probes targeting the COWP gene will discriminate between genotypes 1 and 2 of C. parvum based on single base pair mismatches.

TABLE 7
Specific sequences of Giardia genotyping primers
and probes within the 411-485 bp region
of the β-giardin gene.

| | β-giardin P434 | | SEQ ID |
|---|---|---|---|
| Oligo | Assemblage | Sequence (5′ to 3′) | NO |
| F | A | cctcaagagcctgaacgatctc | 13 |
| | B | cctcaagagcctgaacgacctc | 19 |
| R | A | agctggtcgtacatcttcttcctt | 14 |
| | B | agctggtcatacatcttcttcctc | 20 |
| P | A | ttctccgtggcaatgcccggtct | 15 |
| | B | ttctccgtggcgatgcctgtct | 21 |

Forward (F);
Reverse (R);
Probe (P)

TABLE 8
Genotype detection using β-giardin P434 compared to
recognition of all Giardia tested by β-giardin P241.

Ct values with
Specific Probes

| | | P434 |
|---|---|---|
| Source of Giardia | P241 | (Assemblage A) |
| G. lamblia | | |
| WB | 28.11 | 25.58 |
| H3 | 25.95 | No Ct |
| G-A Stool Isolate | 27.21 | 27.58 |
| G. muris | 23.32 | No Ct |

Assemblage A genotypes: WB, GA stool isolate

Assemblage B genotypes: H3

The p434 primer probe set was used to genotype the Giardia positive stool specimens into assemblage A and B (Table 9). The majority of the samples were of assemblage B, (human genotype) and three mixed infections of assemblages A and B were also observed (Table 9). The two major assemblages of Giardia were also detected in raw sewage samples; assemblage B was the predominant genotype (Table 10).

TABLE 9
Major Genotype Detection of G. lamblia in Stool.

Number of Cysts

| Stool Specimen | Assemblage A^{a} | Assemblage B^{b} |
|---|---|---|
| A | 0 | 11,558 |
| B | 6,331 | 1,034 |
| C | 0 | 1,428 |
| D | 0 | 2,068 |
| E | 0 | 27,218 |
| F | 69 | 118,035 |
| G | 1,262 | 0 |
| H | 0 | 4,852 |
| I | 0 | 3,916 |
| J | 40,530 | 781 |
| K | 0 | 34,081 |
| L | 0 | 352 |
| M | 0 | 456 |
| N | 5,593 | 0 |

^{a}Detection of Giardia using the P434 P-1 (assemblage A) sequence of primers-probe.
^{b}Detection of Giardia using the P434 H3 (assemblage B) sequence of primers-probe.

TABLE 10
Major Genotype Detection of G. lamblia in Raw Sewage.

| | Number of G. lamblia Cysts | |
|---|---|---|
| Sample | Assemblage A^{a} | Assemblage B^{b} |
| Negative Control | 0 | 0 |
| Auteuil 1 | 496 | 5146 |
| Auteuil 2 | 2476 | 8340 |
| Auteuil 3 | 5672 | 7736 |
| Fabreville 1 | 838 | 1815 |
| Fabreville 2 | 2196 | 3663 |
| Fabreville 3 | 545 | 3331 |

^{a}Detection of Giardia using the P434 P-1 (assemblage A) sequence of primers-probe.
^{b}Detection of Giardia using the P434 H3 (assemblage B) sequence of primers-probe. The Auteuil and Fabreville treatment facilities, Laval, Quebec.

### Example 11

Removal of PCR Inhibitors from Environmental Samples

PCR Inhibitor Removal:

Concentration of 2 L water samples resulted in inhibition of real-time PCR. Addition of BSA (Fraction V, SIGMA) at a final concentration of 100 μg/ml or milk powder at a concentration of 2mg/ml resulted in the removal of the inhibitors from 3 out of 4 water bodies tested. Samples from 1 lake were completely inhibitory to real-time PCR in the presence of BSA and required additional steps to remove inhibitors. Additional inhibition removal was carried out during concentration of water samples and DNA extraction. Following filtration of 2 L of water through the 3 μm cellulose nitrate filter, the filter was treated with 20 ml of 0.5 M EDTA pH 8.0 for 5 min then washed with dd water. After washing cysts/oocysts from the filter (described in example 3) the following inhibitor removers were added to the sample in ATL buffer: Chelex® (BIO RAD) slurry, to a final concentration of 20% and PVP-360 (ICN, Aurora, Ohio), to a final concentration of 2%. The samples were incubated for 30 min at 56° C., subjected to freeze/thaw and sonication and centrifuged at 12,000×g for 10 min. The supernatant was processed on a DNeasy column following the manufacturer's description and eluted from the column in 50 μl of dd water.

To detect the presence of inhibitors, environmental sample extracts were spiked with a known concentration of DNA and the Ct values from real-time PCR were compared to the same concentration spiked into dd water (Table 11). The addition of BSA to the PCR mix was sufficient to remove inhibitors from concentrated Heart Lake water samples, enabling amplification of spiked DNA in real-time PCR. BSA did not remove inhibitors from Professor lake samples, however following treatment with EDTA, Chelex® 100 and PVP-360, DNA amplified from Professor Lake with Ct values equivalent to dd water (Table 11).

A strategy involving the addition of EDTA, Chelex® 100 and PVP-360 treatment during DNA extraction, with the addition of BSA in the real-time PCR mastermix can be applied routinely to all environmental samples when large volumes of water are analyzed. These procedures are applicable to other samples such as food and soil. The Mo Bio kit (MO BIO Laboratories Inc., Carlsberg, Calif.) and QIAamp® DNA stool kit (Qiagen) were also effective for inhibitor removal from environmental water samples and may be used under certain conditions. An internal control can be incorporated into the assays, based on a set of template/primers/probe distinct from all the target sequences described herein. Inclusion of an internal positive control to all real-time PCR reactions will indicate the presence of PCR inhibitors.

TABLE 11
Removal of Inhibitors from Environmental Water Samples

Probe

| | β-giardin | COWP |
|---|---|---|
| Sample | Ct | Ct |
| dd Water | 24.88 ± 0.69 | 27.36 ± 0.40 |
| Professor Lake | | |
| Untreated | No Ct | No Ct |
| Treated 1 | 24.89 ± 0.13 | 27.61 ± 0.19 |
| Treated 2 | 25.15 ± 0.94 | 27.99 ± 0.60 |
| Heart Lake | | |
| Untreated | 23.98 ± 0.09 | 27.09 ± 0.35 |
| Treated | 24.34 ± 0.89 | 26.70 ± 0.89 |

Real-time PCR amplification of 500 pg Giardia (β-giardin) or Cryptosporadium (COWP) DNA in the presence of concentrated (from 2 L) environmental water samples. 100 μg/ml BSA in real-time PCR mix Treated samples: 0.5M EDTA. PVP-360 and Chelex® 100

### Example 12

Overcoming PCR Cross-Contamination

To prevent cross-contamination of PCR products to yield false positives in the laboratory one can adopt the use of dUTP and uracil-N-glycosyalse (UNG). In PCR reactions dUTP becomes incorporated into the growing amplicon, rather than dTTP. At the onset of each PCR reaction a UNG treatment to cleave the uracil base from the phosphodiester DNA backbone, thus, rendering the DNA unsuitable for replication, but leaving the thymine-containing sample DNA unharmed (Longo et al., 1990).

### Example 13

Detection of Viable Cells

The present methodology can also be adapted to yield results for only viable cells in a sample. In particular, the presence of RNA in bacterial cells may serve as an indicator of viability, providing that the specific RNA is present only in viable cells and is degraded rapidly upon cell death. A number of studies have focused on nucleic acids associated with VBNC cells as indirect measure of cell viability (reviewed in McDougald et al., 1998). Reverse transcriptase-polymerase chain reaction assays have been developed for the detection of L. monocytogenes (Klein and Juneja, 1997), V. cholerae (Bej et al., 1996), Mycobacterium tuberculosis (Pai et al., 2000), Staphylococcus aureus and E. coli (McKillip et al., 1998)., E. coli O157:H7 (Yaron and Matthews, 2002). Thus, presence of specific mRNA can serve as an indicator of metabolic activity in non culturable cells and may aid in supporting the hypothesis of VBNC.

Another approach to detecting only viable targets by PCR is DNase treatment of the bacterial cells, prior to cell lysis and DNA extraction, to rid the sample of surrounding DNA, and ensure that all DNA detected is from viable cells (Lyon, 2001). For bacterial samples use of irreversible nucleic acid binding dyes that permeates dead cells, such as ethidium nomonoazide (EMA), could facilitate the reduction of background fluorescence signal from the DNA of dead cells (Rudi, 2002).

Viability measurements using ethidium monoazide (EMA) (Molecular Probes, Eugene, Oreg.) treatment were carried out by the following procedure. One milliliter of 100 μg/ml EMA in ddH₂O was added to the bacteria concentrated onto filters in a vacuum filtration unit. The unit was placed in the dark for 5 min to allow the EMA to penetrate into the cells then exposed for 2.5 min to light from a 100 watt halogen light source (Oriel Inc) at a distance of 20 cm, to photo-activate the EMA. After light exposure the filters were washed with 50 ml of ddH₂O, DNA was extracted and qPCR performed. A significant reduction in DNA amplification was observed when bacteria were treated at 100° C. for 20 min then treated with EMA compared with EMA treatment of live cells (Table 12).

TABLE 12
EMA treatment for Viability Determination

qPCR Amplification
of DNA
EMA Treatment

| Bacteria | 0 μg/ml | 100 μg/ml |
|---|---|---|
| Live | +++ | +++ |
| Dead* | +++ | − |

*Dead cells were obtained by treating E. coli for 20 min at 100° C.

A second approach involves treating the samples with EDTA to chelate out divalent cations from dead cells. This allows the collected cells to be treated with Dnase and selectively degrade dead-cell DNA. PCR amplification will occur only from viable cells.

Bacteria concentrated on the filter membranes were treated for 5 min with different concentrations of EDTA: 2 mM, 0.2 mM and 0.02 mM. Following treatment, the filters were washed with 50 ml of ddH₂O, treated for 5 min. with 10 units/ml of the Dnase (RQ1) and washed with 50 ml of water. qPCR was performed using DNA extracted from the treated cells.

### Example 14

Detection of Giardia and Cryptosporidium in Sstool Specimens.

The qPCR assay was used to detect the protozoan pathogens in clinical stool specimens. Giardia was detected, using qPCR, in 16 clinical stool samples that were positive for Giardia as determined by using an immunofluorescence assay performed by the Ontario Ministry of Health parasitology Lab (Table 13). The positive specimens ranged from very low to very high levels of cysts in each patient's stool sample. The qPCR assay using the COWP primer-probe set did not detect Cryptosporidium in the Giardia positives samples. One stool specimen that was positive for Cryptosporidium using IFA was also positive for Cryptosporidium using qPCR, however, no Giardia were present in this sample. Thirty-six stool specimens were negative for both Giardia and Cryptosporidium as determined by both qPCR and IFA. No false positives or false negatives were observed in any of the stool specimens demonstrating the specificity and sensitivity of the qPCR assays for detecting the target pathogens.

TABLE 13
Real-time PCR Detection of Giardia and Cryptosporidium
in Clinical Stool Specimens.

qPCR Detection
(#positive/total samples)

| Stool Specimens* | Giardia | Cryptosporidium |
|---|---|---|
| Giardia and Cryptosporidium | 0/36 | 0/36 |
| Negative | | |
| Giardia Positive | 16/16 | 0/16 |
| Cryptosporidium Positive | 0/1 | 1/1 |

*The presence or absence (positive/negative) of Giardia and Ctyptosporidium in the stool specimens was determined by the MOH and Mount Sinai TML parasitology laboratories, using an immunofluorescence assay (IFA).

### Example 15

Detection of Giardia and Cryptosporidium in Raw Sewage.

The qPCR assay was applied to detection of Giardia and Cryptosporidium in IL raw sewage samples. The results were compared to detection of these pathogens using immunofluorescence assay (IFA). Giardia cysts were detected by qPCR at similar concentrations to IFA (Table 14). No Cryptosporidium oocysts were detected by either method, suggesting that the oocysts were absent or present in low numbers below our detection limit.

TABLE 14
Comparison of qPCR and IFA for Detection of G. lamblia and
Cryptosporidium in 1 L Sewage Samples.

| | |
|---|---|
| Number of | Number of |
| G. lamblia | C. parvum |
| Cysts | Oocysts |

| Sample^{a} | qPCR | IFA | qPCR | IFA |
|---|---|---|---|---|
| Negative Control | 0 | — | 0 | — |
| Auteuil 1 | 5642 | 2380 | 0 | 0 |
| Auteuil 2 | 10816 | 9880 | 0 | 0 |
| Auteuil 3 | 13408 | 7980 | 0 | 0 |
| Fabreville 1 | 2653 | 9900 | 0 | 0 |
| Fabreville 2 | 5859 | 6660 | 0 | 0 |
| Fabreville 3 | 3876 | 4290 | 0 | 0 |

^{a}NC, negative control in qPCR; The Auteuil and Fabreville treatment facilities, Laval, Quebec.

### Example 16

Detection of Bacteria on Carcass and Environmental Swabs:

Direct Detection of Bacteria on Sponges: We have tested the use of a pulsifier (Microgen Bioproducts) for its ability to dislodge bacteria from the sponge matrix and allow detection of bacteria using the qPCR assay. The pulsifier was selected over use of a stomacher because of the efficiency of the pulsifier to detach bacteria from a matrix while causing minimal disruption of the matrix (Kang and Dougherty, 2001). Results obtained using the pulsifier for direct detection of bacteria spiked onto sponges demonstrated that greater than 70% of spiked cells were recovered when cells were spiked onto either dry sponges or sponges hydrated with buffered peptone water (Table 15). In addition, as few as 50 E. coli O157:H7 cells that were spiked onto sponges were detected.

TABLE 15
qPCR detection of E. coli 0157: H7 spiked onto sponges.

| Number of Bacteria | % Recovery |
|---|---|
| spiked onto | of Bacteria |
| Sponges | from Sponges* |
| Dry Sponge | |
| 500 | 70 |
| 50 | 78 |
| Buffered Peptone | |
| Water Sponge | |
| 500 | 150 |
| 50 | 82 |

*Percent recovery is based on the qPCR detection of bacteria spiked onto sponges then collected on a filter compared with bacteria spiked directly onto filters (positive control).

Selection of carcass swab sponges and hydration buffer: Research in the late 1980's demonstrated that certain sponge types are inhibitory to growth of bacteria in culture (Llabres and rose, 1989). Currently, all sponges for use in bacterial detection from carcass swabs are tested to ensure they are “biocide” free, for use in detection of bacteria by cultivation. These sponges have not been tested for their suitability for use in qPCR. We conducted a study to determine whether the cellulose sponges sold by Bio International Inc. were inhibitory to qPCR. For these assays, sponges were placed in water containing 0.025% Tween 20, pulsified to dislodge material from the sponges and the homogenate collected by vacuum filtration onto filter membranes. The concentates on the filters were extracted using Ultraprepman (ABI) extraction solution and assayed for inhibition in the qPCR assay by determining the efficiency of amplification of a known amount of purified DNA in the presence of the extracts compared to the presence of water. The dry sponges were not qPCR inhibitory, whereas, the neutralizing buffer used in environmental swabs was completely inhibitory to the qPCR table 16). Washing the neutralizing buffer sponges overnight in ddH₂O removed the qPCR inhibitory effect (Table 16).

There buffers, commonly used to hydrate sponges for wet-swabbing of carcases, were tested to ensure the buffers were not inhibitory to qPCR. Butterfield's buffer, Letheen's broth and phosphate buffered peptone water were compared to hydration with ddH₂O. None of the buffers were inhibitory to qPCR when added directly to the qPCR assay at a volume of 5 μl (data not shown). No difference in the Ct value was observed in the detection of DNA spiked into the PCR assay when the different buffers were compared to the ddH₂O control, indicating that none of the buffers used to hydrate the sponges were inhibitory to the qPCR assay (Table 17). The qPCR assay can be used for detection of bacteria on sponges hydrated in either Letheen's, Butterfield's or buffered peptone water.

TABLE 16
qPCR detection of E. coli 0157:H7 DNA spiked
into the PCR assay in the presence of extracts
from different types of sponges.

| Sponge Type | Ct* ± SD |
|---|---|
| None | 22.11 ± 0.18 |
| Neutralizing Buffer | No Ct |
| Washed Neutralizing Buffer | 22.84 ± 0.32 |
| Dry | 22.55 ± 0.49 |
| Washed Dry | 23.05 ± 0.66 |

*Ct, cycle threshold = cycle at which the detection crosses the baseline fluorescence.
A Ct value indicates the presence of target DNA.
No Ct indicates that no specific target DNA was present.

TABLE 17
Comparison of qPCR detection of DNA spiked into the
PCR assay in the presence of extracts from the sponges
hydrated in different buffers.

| Buffer used to Hydrate | Detection of Spiked DNA |
|---|---|
| Dry Sponges* | Ct ± SD |
| H₂0 | 28.55 ± 0.49 |
| Butterfields | 28.94 ± 0.47 |
| Letheen's | 28.74 ± 0.67 |
| Buffered Peptone Water | 29.08 ± 0.66 |

*10 ml of each buffer was used to hydrate sponges. The sponges were pulsified in 0.025% Tween water and the homogenate was concentrated through a membrane filter using vacuum filtration. Concentrated material was extracted from the filter using Ultraprepman (ABI). Each qPCR well contained 5 ul of the extracted material.

Collection and Concentration of Bacteria from Sponge Swabs after Growth in Enrichment Media:

Filter washes for media from enrichment: The following were tested to work out the optimal washes, Inhibitex Tablets from a Qiagen stool kit, PVP 40 (polyvinylpyrrolidone), EDTA (0.5 M), ETOH (25%) and MQ Water Alone.

Effects of Washes on Spent Media Inhibition:

A) Our results suggest that a 25% ETOH wash followed by water eliminated the inhibition with a 10 ml sample, and with a 25-ml sample. 50-ml samples collected still were inhibitory (Table 18).

TABLE 18
A comparison of spent and fresh media with different washes

| Sample | Wash Treatment | CT values(Ct ± SD) |
|---|---|---|
| 1. Positive control | 10 ml water, 100 000 cells, | 22.59 ± 0.52 |
| | 20 ml water wash | 21.88 ± 0.32 |
| 2. Negative control | 10 ml fresh media, 100 000 | No ct |
| | cells, 40 ml water | No ct |
| 3. Experimental | 10 ml fresh media, 100 000 | 25.01 ± 0.52 |
| | cells, 10 ml each EDTA, | 29.69 ± 0.43 |
| | ETOH, 20 ml water | |
| 4. Negative control | 10 ml spent media + 10 ml | No ct |
| | PVP, EDTA, ETOH, 20 ml | No ct |
| | water | |
| 5. Experimental | 10 ml spent media, 100 000 | No ct |
| | cells, 10 ml PVP, EDTA, | No ct |
| | ETOH, 20 ml water | |
| 6. Experimental | 10 ml spent media, 100 000 | 22.57 ± 0.21 |
| | cells, 10 ml each EDTA, | 24.69 ± 0.21 |
| | ETOH, 20 ml water | |
| 7. Experimental | 10 ml spent media, 100 000 | 20.09 ± 0.34 |
| | cells, 10 ml ETOH, 20 ml | 21.19 ± 0.43 |
| | water | |
| 8. Experimental | 10 ml spent media, 100 000 | No ct |
| | cells, 10 ml EDTA, 20 ml | No ct |
| | water | |
| 9. Experimental | 10 ml spent media, 100 000 | No ct |
| | cells, 50 ml water | No ct |

B) Our results suggest that 35 ml media (TSB) with 500 cells on sponge with of 25 ml ETOH and 100 ml of water washes gave a good Ct value (Table 19)

TABLE 19
Time points for enrichment of media with sponges

| | | CT value | CT value | CT value |
|---|---|---|---|---|
| Sample | Wash Treatment | 4 hr | 5 hr | 6 hr |
| 1. Positive | 25 ml water, 100,000 cells, | — | 21.67 ± 0.52 | 22.17 ± 0.43 |
| control | 50 ml water wash | | | |
| 2. Media control | 35 ml media, 25 ml ETOH | No ct | No ct | No ct |
| | and 100 ml of water | | | |
| 3. Experimental | 35 ml media + 500 cells on | 23.54 ± 0.52 | 22.72 ± 0.43 | 21.76 ± 0.53 |
| | sponge, 25 ml ETOH, 100 ml | | | |
| | water | | | |

Protocol for measuring from samples:

1. Sponge swabs will be put into 125 ml nutrient broth or TSB media, and incubated at 37° C.

2. At some time point 2-5 hours after incubation, the media will be divided into three aliquots, 25 ml for culturing, and up to 50 ml for qPCR

3. The procedure for washing and collection is described above.

Although the foregoing invention has been described in some detail by way of illustration and examples for the purposes of clarity, one skilled in the art will appreciate that certain changes and modifications may be practiced within the scope of the invention as defined by the appended claims.

### References

Articles referenced herein, and incorporated herein by reference, are listed below:

1. American Public Health Association. 1995. Standard Methods for the Examination of Water and Wastewater. Washington, D.C.

2. Acheson, D. W. K. 2000. How does Escherichia coli O157:H7 testing in meat compare with what we are seeing clinically? J. Food Prot. 63:819-821.

3. Andersen, R. J., Luu, H. A., Chen, D. Z. X., Holmes, C. F. B., Kent, M. L., LeBlanc, M. and Taylor, F. J. R. (1993) Chemical and biological evidence links microcystins to salmon ‘netpen liver disease’. Toxicon 31: 1315-23.

4. Apte, S. C., Davies, C. M. and Peterson, S. M. 1995. Rapid detection of faecal coliforms in sewage using a colorimetric assay of β-D-galactosidase. Wat. Res. 29: 1803-6.

5. Bej, A. K., DiCesare, J. L., Haff, L. and Atlas, R. M. 1991b. Detection of Escherichia coli and Shigella spp. in water by using the polymerase chain reaction and gene probes for uid. Appl. Environ. Microbiol. 57: 1013-7.

6. Bej, A. K., Mahbubani, M. H., Miller, R., DiCesare, J. L., Hoff, L. and Atlas, R. M. 1990a. Multiplex PCR amplification and immobilized capture probes for detection of bacterial pathogens and indicators in water. Mol. Cell. Probes 4: 353-65.

7. Bej, A. K., McCarty, S. C. and Atlas, R. M. 1991a. Detection of coliform bacteria and Escherichia coli by multiplex PCR: comparison with defined substrate and plating methods for water quality monitoring. Appl. Environ. Microbiol. 57: 2429-32.

8. Bej, A. K., Ng, W. Y., Morgan, S., Jones, D. D. and Mahbubani, M. H. 1996. Detection of viable Vibrio cholerae by reverse-transcriptase polymerase chain reaction (RT-PCR). Mol. Biotech. 5: 1-10.

9. Bej, A. K., Steffan, R. J., DiCesare, J., Haff, L. and Atlas, R. M. 1990b. Detection of coliform bacteria in water by PCR and gene probes. Appl. Environ. Microbiol. 56: 307-14.

10. Beebakhee, G., Louie, M., De Azavedo J., and Brunton, J. 1992. Cloning and nucleotide sequence of the eae gene homologue from enterohemorrahagic Esherichia coli serotype O157:H7. FEMS Microbiol. Lett. 91:63-8.

11. Belosevic, M. Guy, R. A., Kilani, R. T., Neumann, N. F., Gyurek, L. L., Liyanage, L. R. J., Millard, P. J. and Finch, G. R. 1997. Nucleic acid stains as indicators of Cryptosporidium oocyst viability. Int. J. Parasitol. 27:787-8.

12. Bemander R, Palm J E, Svard S G. 2001. Genome ploidy in different stages of the Giardia lamblia life cycle. Cell Microbiol. 3:55-62.

13. Black, E. K., Finch, G. R., Taghi-Kilani, R. and Belosevic, M. 1996. Comparison of assays for Cryptosporidium parvum oocyst viability after chemical disinfection. FEMS Microbiol. Lett. 135:187-9.

14. Bury, N. R., McGeer, J. C., Eddy, F. B. and Codd, G. A. 1997. Liver damage in brown trout, Salmo trutta L., and rainbow trout, Oncorhyncus mykiss (Walbaum) following administration of the cyanobacterial hepatotoxin microcystin-LR via the dorsal aorta. J. Fish Dis. 20: 209-215.

15. Call, D. R., Brockman, R. J., and Chandler, D. P. 2001. Detecting and genotyping Escherichia coli O157:H7 using multiplexed PCR and nucleic acid microarrays. International J. Food Microbiol. 67: 71-80.

16. Chen, X-M., Keithly, J. S., Paya, C. V. and LaRusso, N. F. 2002. Cryptosporidiosis. N. Engl. J. Med.346:1723-31.

17. Chu F. S., Huang X. and Wei R. D. 1989. Enzyme-linked immunosorbent assay for microcystins in blue-green algal blooms. J Assoc Off Anal Chem 73:451-456.

18. Chung, E., Aldom, J. E., Chagala, A. H., Kostrzynska, M., Lee, H., Palmateer, G., Trevors, J. T., Unger, S. and de Grandis, S. 1998. Detection of Cryptosporidium parvum oocysts in municipal water samples by the polymerase chain reaction. J. Microbiol. Meth. 33:171-80.

19. Codd, G. A. 1994. Blue-green algal toxins: water-borne hazards to health. In Water and Public Health ed. Golding, A. M. B, Noah N., and Stanwell-Smith, R. pp. 271-278. London: Smith-Gordon.

20. Codd, G. A. 1995. Cyanobacterial toxins: occurrence, properties and biological significance. Wat. Sci. Tech. 32: 149-56.

21. Codd, G. A., Jefferies, T. M., Keevil, C. W. and Potter E., eds. 1994. Detection Methods for Cyanobacterial Toxins. The Royal Society of Chemistry, Cambridge, 191 pp.

22. Deng, M. Y. and Frantamico, P. M. 1996. A multiplex PCR for rapid identification of shiga-like toxin-producing Escherichia coli O157:H7 isolated from foods. J. Food Protect. 59: 570-76.

23. Desmarchelier, P. M., Bilge, S. S., Fegan, N., Mills, L. Jr, Vary, J. C. and Tarr, P. I. 1998. A PCR specific for Escherichia coli O157 based on the rfb locus encoding O157 lipopolysaccharide. J. Clinl. Microbiol. 36: 1801-4.

24. Di Giovanni, G. D., Hashemi, F. H., Shaw, N. J., Abrams, F. A., LeChevalier, M. W. and Abbaszadegan, M. 1999. Detection of infectious Cryptosporidium parvum oocysts in surface and filter backwash water samples by immunomagnetic separation and integrated cell culture-PCR. Appl. Environ. Microbiol. 65:3427-32.

25. Dillingham, R. A., Lima, A. A., Guerrant, R. L. 2002. Cryptosporidiosis: epidemiology and impact. Microb. Inf. Dis. 4:1059-66.

26. Doyle, M. P. and Schoeni, J. L. 1984. Survival and growth characteristics of Escherichia coli associated with hemorrhagic colitis. Appl Environ Microbiol 48:855-856.

27. DuPont, H. L., Chappell, C. L., Sterling, C. R., Okhuysen, P. C., Rose, J. B. and Jakubowski, W. 1995. The infectivity of Cryptosporidium parvum in healthy volunteers. N. Engl. J. Med. 332:855-9.

28. Farmer, J. J. and Davis, B. R. 1985. H7 antiserum-sorbitol fermentation medium: a single-tube screening medium for detecting Escherichia coli O157:H7 associated with hemorrhagic colitis. J Clin Microbiol 22:620-625.

29. Feng, P., Day, M., Abe, A. and Takeda, T. 2001a. Isogenic strain of Escherichia coli O157:H7 that has lost both Shiga toxin 1 and 2 genes. Clin. Diag. Lab. Imm. 8: 711-7.

30. Feng, P., S. Weagant and S. Monday. 2001b. Genetic analysis for virulence factors in Escherichia coli O104:H21 that was implicated in an outbreak of hemorrhagic colitis. J. Clin. Microbiol. 39:24-28.

31. Fields, P. I., Blom, K., Hughes, H. J., Helsel, L. O., Feng, P. and Swaminathan, B. 1997. Molecular characterization of the gene encoding H antigen in Escherichia coli and development of a PCR-restriction fragment length polymorphism test for identification of E. coli O157:H7 and O157:NM. J. Clin. Microbiol. 35: 1066-70.

32. Fontaine, M and Guillot, E. 2002. Development of a TaqMan quantitative PCR assay specific for Cryptosporidium parvum. FEMS Microbiol. Letts. 214: 13-7.

33. Frantamico, P. M., Sackitey, S. K., Wiedmann, M. and Deng,M.Y 1995 Detection of Escherichia coli O157:H7 by multiplex PCR. J. Clin. Microbiol. 33: 2188-91.

34. Gannon, V. P., D'Souza, S., Graham, T., King, R. K., Rahn, K. and Read, S. 1997. Use of the flagellar H7 gene as a target in multiplex PCR assays and improved specificity in identification of enterohemorrhagic Escherichia coli strains. J. Clin. Microbiol. 35:656-62.

35. Gannon, V. P. J., King, R. K., Kim, J. Y. and Thomas, E. J. G. 1992. Rapid and sensitive method for detection of Shiga-like toxin-producing Escherichia coli in ground beef using the polymerase chain reaction. Appl. Environ. Microbiol. 58: 3809-15.

36. Gannon, V. P. J., Rashed, M., King R. K., and Golsteyn Thomas, E. J. 1993. Detection and characterization of the eae gene of Shiga-like toxin-producing Esherichia coli using polymerase chain reaction. J. Clin. Microbiol. 31: 1268-74.

37. Germani, Y., Begaud, E. and Le Bouguenec, C. 1997. Detection of the Escherichia coli attaching and effacing gene (eaeA) in enteropathogenic strains by polymerase chain reaction. Res. Microbiol. 148, 177-181.

38. Gobet, P., Buisson, J. C., Vagner, O., Naciri, M., Grappin, M., Comparot, S., Harley, G., Aubert, D., Varga, I., Camerlynck, P. and Bonnin, A. 1997. Detection of Cryptosporidium parvum DNA in formed human feces by a sensitive PCR-based assay including uracil-N-glysocsylase inactivation. J. Clin. Mircobiol. 35:154-6.

39. Heitman, T. L., Frederick, L. M., Viste, J. R., Guselle, N. J., Morgan, U. M., Thompson, R. C. A. and Olsen, M. E. 2002. Prevalence of Giardia and Cryptosporidium and characterization of Cryptosporidium spp. Isolated from wildlife, human, and agricultural sources in the North Saskatchewan river basin in Alberta, Canada. Can. J. Microbiol. 48:530-41.

40. Higgins, J. A., Fayer, R., Trout, J. M. Xiao, L., Lal, A. A., Kerby, S. and Jenkins, M. C. 2001. Real-time PCR for the detection of Cryptosporidium parvum. J. Microbiol. Meth. 47:323-7.

41. Holberton, D. V. and J. Marshall. 1995. Analysis of consensus sequence patterns in Giardia cytoskeleton gene promoters. Nucl. Acids Res. 23:2945-2953.

42. Jackson, M. P., Neill, R. J., O'Brien, A. D., Holmes, R. K. and Newland, J. W. 1987. Nucleotide sequence analysis and comparison of the structural genes for shiga-like toxin I and shiga-like toxin II encoded by bacteriophages from Escherichia coli 933. FEMS Microbiol. Lett. 44, 109-14.

43. Jenkins, M. C., Trout, J., Abrahamsen, M. S., Lancto, C. A., Higgins, J. and Fayer, R. 2000. Estimating viability of Cryptosporidium parvum oocysts using reverse transcritase-polymerase chain reaction (RT-PCR) directed at mRNA encoding amyloglucosidase. J. Microbiol. Meth. 43:97-106.

44. Jerse, A. E., Yu, J., Tall, B. D., and Kaper, J. B. 1990. A genetic locus of enteropathogenic Esherichia coli necessary for the production of attaching and effacing lesions on tissue culture cells. PNAS 87:7839-43.

45. Johnson, D. W., Pieniazek, N. J., Griffin, D. W., Misener, L. and Rose, J. B. 1995. Development of a PCR protocol for sensitive detection of Cryptosporidium oocysts in water samples. Appl. Env. Microbiol. 61:3849-55.

46. Josephson, K. L., Gerba, C. P. and Pepper, I. L. 1993. Polymerase chain reaction detection of nonviable bacterial pathogens. Appl. Environ. Microbiol. 59 , 3513-3515.

47. Jothikumar, N. and Griffiths, M. W. 2002. Rapid detection of Escherichia coli O157:H7 with multiplex real-time PCR assays. Appl Env Microbiol 68: 3169-71.

48. Karch H., Meyer T., Russmann H. and Heesmann J. 1996. Frequent loss of shiga like toxin genes in clinical isolates of Escherichia coli upon subcultivation. Infec. Immun. 60: 3464-7.

49. Karmali, M. A. 1989. Infection by verocytotoxin-producing Escherichia coli. Clin. Microbiol. Rev. 2: 15-38.

50. Kaucner, C. and Stinear, T. 1998. Sensitive and rapid detection of viable Giardia cysts and Cryptosporidium parvum oocysts in large-volume water samples with wound fiberglass cartridge filters and reverse transcription-PCR. Appl. Environ. Microbiol. 64:1743-9.

51. Kim, Y. B., Okuda, J., Matsumoto, C., Morigaki, T., Asai, N., Watanabe, H. and Nishbuchi, M. 1998. Isolation of an Escherichia coli O157:H7 strain producing Shiga toxin 1 but not Shiga toxin 2 from a patient with hemolytic uremic syndrome in Korea. FEMS Microbiol Lett 166:43-8.

52. Klein, P. G. and Juneja, V. K. 1997. Sensitive detection of viable Listeria monocytogenes by reverse transcription-PCR. Appl. Environ. Microbiol 63: 4441-8.

53. Kuhn, R. C. and Oshima, K. H. 2001. Evaluation and optimization of a reusable hollow fiber ultrafilter as a fist step in concentrating Cryptosporidium parvum oocysts from water. Wat. Res. 35: 2779-83.

54. Kushner, S. R. 1996. mRNA decay. In Escherichia coli and Salmonella Cellular and Molecular Biology ed. Neidhardt, F. C. pp. 849-860. Washington, USA: ASM Press.

55. Laberge I., Ibrahim, A., Barta, J. R. and Griffiths, M. W. 1996. Detection of Cryptosporidium parvum in raw mile by PCR and oligonucleotide probe hybridization. Appl. Environ. Microbiol. 62:3259-64.

56. Limor, J. R., Lal. A. A and Xiao, L. 2002. Detection and differentiation of Cryptosporidium parasites that are pathogenic for humans by real-time PCR. J. Clin. Microbiol. 40:2335-8.

57. Longo, M. C., Berninger, M. S. and Hartley J. L. 1990. Use of uracil DNA glycosylase to control carry-over contamination in polymerase chain reactions. Gene 93: 125-8.

58. Louie, M., De Azavedo, J., Clarke, R., Lior, H., Richter, M., and Brunton, J. 1994. Sequence heterogeneity of the eae gene and detection of vertoxin producing Esherichia coli using serotype-specific primers. Epidemiol. Infec. 112: 449-61.

59. Louie, M., De Azavedo, J., Handelsman, M. Y. C., Clark, C. G., Ally, D., Dytoc, M., Sherman, P., and Brunton, J. 1993. Expression and characterization of the eaeA gene product of Esherichia coli serotype O157:H7. Infec. Imm. 61:4085-92.

60. Lyon, W. 2001. TaqMan PCR for detection of Vibrio cholerae O1, O139, non-O1, and non-O139 in pure cultures, raw oysters, and synthetic seawater. Appl. Env. Microbiol. 67: 4685-93.

61. MacDonald, L. M., Sargent, K., Armson, A., Thompson, R. C. A. and Reynoldson, J. A. 2002. The development of a real-time quantitative-PCR method for characterization of a Cryptosporidium parvum in vitro culturing system and assessment of drug efficacy. Mol. Biochem. Parasitol. 121:279-82.

62. Mahbubani, M. H., Bej, A. K., Perlin, M. H., Schaeffer III, F. W., Jakubowski, W. and Atlas, R. M. 1991. Detection of Giardia cysts by using the polymerase chain reaction and distinguishing live from dead cysts. Appl. Environ. Microbiol. 57:3456-61.

63. Mahbubani, M. H., Schaefer III, F. W., Jones, D. D. and Bej, A. K. 1998. Detection of Giardia in environmental waters by immuno-PCR amplification methods. Curr. Microbiol. 36:107-13.

64. Masters, C. I., Shallcross, J. A. and MacKey, B. M. 1994. Effect of stress treatments on the detection of Listeria monocytogenes and enterotoxigenic Escherichia coli by the polymerase chain reaction. J App Bacteriol 77: 73-9.

65. Mayer, C. L. and Palmer, C. J. 1996. Evaluation of PCR, nested PCR and Fluorescent antibodies for detection of Giardia and Cryptosporidium species in wastewater. Appl. Environ. Microbiol. 62: 2081-5.

66. McDougald, D., Rice, S. A., Weichart, D., Kjelleberg, S. 1998. Nonculturability: adaptation or debilitation? FEMS Microbiol Ecol 25: 1-9.

67. McKillip, J. L., Jaykus, L. A. and Drake, M. 1998. rRNA stability in heat-killed and UV-irradiated enterotoxigenic Staphylococcus aureus and Escherichia coli O157:H7. Appl. Environ. Microbiol. 64: 4264-68.

68. Meng, J., Zhao, S., Doyle, M. P., Mitchell, S. E. and Kresovich, S. 1997. A multiplex PCR for identifying shiga-like toxin producing Escherichia coli O157:H7. Lett. Appl. Microbiol. 24: 172-76.

69. MMWR. 2002. Manufacturer's recall of rapid assay kits based on false positive Cryptosporidium anitgen tests—Wisconsin 2001-2002. Morb Mortal Wkly Rep. 51: 189.

70. Morgan, U. M., Pallant, L., Dwyer, B. W., Forbes, D. A., Rich, G. and Thompson, R. C. A. 1998. Comparison of PCR and microscopy for detection of Cryptosporidium parvum in human fecal specimens: clinical trial. J. Clin. Microbiol. 36:995-8.

71. Neumann, N. F., Gyruk, L. L., Gammie, L., Finch, G. R., and Belosevic, M. 2000. Comparison of animal infectivity and nucleic acid staining for assessment of Cryptosporidium parvum viability in water. Appl. Environ. Microbiol. 66:406-12.

72. Nishizawa, T., Ueda, A., Asayama, M., Fujii, K., Harada, K., Ochi, K. and Shirai, M. 2000. Polyketide synthase gene coupled to the peptide synthetase module involved in the biosythesis of the cyclic heptapeptide microcystins. J Biochem. 127: 779-89.

73. Nishizawa, T., Asayama, M., Fuigii, K., Harada, K. and Shirai, M. 1999. Genetic analysis of the peptide synthetase genes for a cyclic heptapeptide microcystin in Microcystis spp. J. Biochem. 126: 520-29.

74. Oberst, R. D., Hays, M. P., Bohra, L. K., Phebus, R. K., Yamashiro, C. T., Paszko-Kolva, C., Flood, S. J. A., Sargeant, J. M., and Gillespie, J. R. 1998. PCR-based DNA amplification and presumptive detection of Esherichia coli O157:H7 with an internal fluorogenic probe and the 5′ nuclease (TaqMan) assay. Appl. Environ. Microbiol. 64: 3389-96.

75. Olsen, M. E., Thorlakson, C. L., Deselliers, L., Morck, D. W. and McAllister, T.A. 1997. Giardia and Cryptosporidium in Canadian farm animals. Vet. Parasitol. 68:375-81.

76. Pai S R, Actor J K, Sepulveda E, Hunter R L Jr, Jagannath C. 2000. Identification of viable and non-viable Mycobacterium tuberculosis in mouse organs by directed RT-PCR for antigen 85B mRNA. Microb Pathog. 28:335-342.

77. Patel, S., Pedraza-Diaz, S. and McLauchlin, J. 1999. The identification of Cryptosporidium species and Cryptosporidium parvum directly from whole faeces by anbalysis of a multiplex PCR of the 18S rRNA gene and by PCR/RFLP of the Cryptosporidium outer wall protein (COWP) gene. Int. J. Parasitol. 29:1241-7.

78. Payment, O., Berte, A., Prévost, M., Ménard, B. and Barbeau, B. 2000. Occurrence of pathogenic microorganisms in the Saint Lawrence River (Canada) and comparison of health risks for populations using it as their source of drinking water. Can. J. Microbiol. 46:565-76.

79. Rendtorff, R. C. 1954. The experimental transmission of human intestinal protozoan parasites. II. Giardia lamblia cysts given in capsules. Am. J. Hyg. 59:209-20.

80. Restaino, L., H. Castillo, D. Stewart, and M. Tortorello. 1996. Antibody-direct epifluorescent filter technique and immunomagnetic separation of 10-h screening and 24-h confirmation of Escherichia coli O157:H7 in beef. J. Food Prot. 59:1072-5.

81. Rice and Schaefer III., 1982. Improved in vitro excystation procedure for Giardia lamblia cysts. J. Clin. Microbiol. 14: 709-10.

82. Rigsbee, W., Simpson, L. M. and Oliver, J. D. 1997. Detection of the viable but nonculturable state in Escherichia coli O157:H7. J Food Saf. 16: 225-62.

83. Rimhanen-Finne, R., Ronkainen, P. and Hanninen, M.-L. 2001. Simultaneous detection of Cryptosporidium parvum and Giardia in sewage sludge by IC-PCR.

84. Rippka, R., Deruelles, J., Waterbury, J., Herdman, M., and Stainer, R. 1979. Generic assignments, strain histories and properties of pure cultures of cyanobacteria. J Gen Microbiol 111: 1-61.

85. Robertson, L. J. and Gjerde B. 2001. Occurrence of parasites on fruits and vegetables in Norway. J. Food Prot. 64: 1793-8.

86. Rochelle, P. A., Ferguson, S. M., Handojo, T. J., De Leon, R., Stewart, M. H. and Wolfe, R. L. 1997. An assay combining cell culture with reverse transcriptase PCR to detect and determine the infectivity of waterborne Cryptosporidium parvum. Appl. Environ. Microbiol. 63: 2029-37.

87. Rochelle, P. A., Marshall, M. M., Mead, J. R., Johnson, A. M., Korich, D. G., Rosen, J. S. and De Leon, R. 2002. Comparison of in vitro cell culture and a mouse assay for measuring infectivity of Cryptosporidium parvum. Appl. Environ. Microbiol. 68:3809-17.

88. Rompré, A., Servais, P., Baudart, J., De-Roubin M-R., and Laurent, P. 2002. Detection and enumeration of coliforms in drinking water: current methods and emerging approaches. J. Microbiol. Meth. 49: 31-54.

89. Rose, J. B. Darbin, H. and Gerba, C. P. 1988, Correlation of the protozoa, Cryptosporidium and Giardia with water quality variables in a watershed. Water Sci. Technol. 20:271-6.

90. Rose, J. B. and Slifko, T. R. 1999. Giardia, Cryptosporidium, and Cyclospora and their impact on foods: a review. J Food Prot 62: 1059-70.

91. Rudi, K., Nogva, H. K., Moen, B., Nissen, H., Bredholt, S., Møretrø, T., Naterstad, K., and Holck, A. 2002. Development and application of new nucleic acid-based technologies of microbial community analyses in foods. Int. J. Food Microbiol. 78:171-80.

92. Rudi, K., Skulberg, O. M., Larsen, F. and Jakobsen, K. S. 1998. Quantification of toxic cyanobacteria in water by use of competitive PCR followed by sequence-specific labeling of oligonucleotide probes. Appl. Environ. Microbiol. 64: 2639-43.

93. Schmidt, H., Plaschke, B., Franke, S., Russmann, H., Schwarzkopf, A., Heesemann, J. and Karch, H. 1994. Differentiation in virulence patterns of Escherichia coli possessing eae genes. Medical Microbiology and Immunology 183: 23-31.

94. Sheppard, K. M. and Wyn-Jones, A. P. 1996. An evaluation of methods for the simultaneous detection of Cryptosporidium oocysts and Giardia cyst from water. Appl. Environ. Microbiol. 62:1317-22.

95. Sharma, V. K., Dean-Nystrom, E. A., and Casey, T. A. 1999. Semi-automated fluorogenic PCR assays (TaqMan) for rapid detection of Esherichia coli O157:H7 and other Shiga toxigenic E. coli. Mol. Cell. Probes 13: 291-302.

96. Simmons, O. D., Sobsey, M. D., Heaney, C. D., Schaefer III, F. W. and Francy, D. S. 2001. Concentration and detection of Cryptosporidium oocysts in surface water samples by method 1622 using ultrafiltratio and capsule filtration. Appl. Environ. Microbiol. 67:1123-7.

97. Sivonen, K. and Jones, G. 1999. Cyanobacterial toxins. In Toxic cyanobacteria in water; a guide to their public heath consequences, monitoring, and management ed. Chorus, I. and Bartram, pp. 41-111. London: J. E&FN Spon, on behalf of WHO.

98. Stinear, T., Matusan, A., Hines, K. and Sandery, M. 1996. Detection of a single viable Cryptosporidium parvum oocyst in environmental water concentrates by reverse transcription-PCR. Appl. Environ. Microbiol. 62:3385-90.

99. Tamani-Shacoori, Z. Jolivet-Gougeon, A., and Cormier, M. 1996. Comparison of direct PCR and PCR amplification after DNA extraction for the detection of viable enterotoxigenic Escherichia coli in laboratory microcosms. J. Microbiol. Meth. 26: 21-6.

100. Tanriverdi, S., Tanyeli, A. Balamisli, F., Koksal, R., Kilinc, F., Feng, X., Batzer, G., Tzipori, S. and Widmer, G. 2002. Detection and genotyping of oocysts of Cryptosporidium parvum by real-time PCR and melting curve analysis. J Clin. Microbiol. 40: 3237-3244.

101. Thompson, R. C. A. 2000. Giardia as a re-emerging infectious disease and its zoonotic potential. Int. J. Parasitol. 30:1259-67.

102. Thompson, R. C. A., Hopkins, R. M. and Homan, W. L. 2000. Nomenclature and genetic groupings of Giardia infecting mammals. Parasitol. Today 16:210-3.

103. Thurston-Enriquez, J. A., Watt, P., Dowd, S. E., Enriquez, R., Pepper, I. L. and Gerba, C. P. 2002. Detection of protozoan parasites and microsporidia in irrigation waters used for crop production. J. Food Protect. 65:378-82.S

104. Tillett D., Dittmann E., Erhard M.,von Döhren H., Börner T., and Neilan B. 2000. Structural organization of microcystin biosynthesis in Microcystis aeruginosa PCC7806: an integrated peptide-polyketide synthetase system. Chem Biol 7: 753-64.

105. U.S. Environmental Protection Agency. 1999. Method 1623: Cryptosporidium and Giardia in water by filtration /IMS/FA. Publication EPA-821-R-99-006.

106. Uyttendaele, M., Bastiaansen, A. and Debevere, J. 1997. Evaluation of the NASBA nucleic acid amplification system for assessment of the viability of Campylobacter jejuni. Int J Food Microbiol 37: 13-20.

107. Uyttendaele, M., van Boxstael, S., and Debevere, J. 1999. PCR assay for detection of the E. coli O157:H7 eae-gene and effect of the sample preparation method on PCR detection of heat-killed E. coli O157:H7 in ground beef. Int. J. Food Microbiol. 52: 85-95.

108. Vanderploeg, H. A., Liebig, J. R., Carmichael, W. W., Agy, M. A. Johengen, T. H., Fahnenstiel, G. L. and Nalepa T. F. 2001. Zebra mussel (Dreissena polymorpha) selective filtration promoted toxic Microcystis blooms in Saginaw Bay (Lake Huron) and Lake Erie. Can J Fish Aq Sci 58, 1208-21.

109. Vesy, C. J. and Peterson, W. L. 1999. Review article: the management of Giardiasis. Aliment. Pharmacol. Ther. 13:843-50.

110. Walker, M. J., Montemagno, C., Bryant, J. C. and Ghiorse, W. C. 1998. Method detection limits of PCR and immunofluorescence assay for Cryptosporidium parvum in soil. Appl. Envir. Microbiol. 64:2281-3.

111. Wallis, P. M., Erlandsen, S. L., Isaac-Renton, J. L., Olsen, M. E., Robertson, W. J. and van Keulen, H. 1996. Prevalence of Giardia cysts and Cryptosporidium oocysts and characterization of Giardia spp. isolated from drinking water in Canada. Appl. Environ. Microbiol. 62:2789-2792.

112. Wawrik B., Paul J. H. and Tabita F. R. 2002. Real-time PCR quantification of rbcL (Ribulose-1,5-bisphosphate carboxylase/oxygenase) mRNA in diatoms and pelagophytes. Appl Env Microbiol 68: 3771-9.

113. Webster, K. A., Smith, H. V., Giles, M., Dawson, L., and Robertson, L. J. 1996. Detection of Cryptosporidium parvum oocysts in faeces: comparison of conventional coprosocpical methods and the polymerase chain reaction. Vet. Parasitol. 61:5-13.

114. Willshaw, G. A., Thirlwell, J., Jones, A. P., Parry, S., Salmon, R. L. and Hickey, M. 1994. Vero cytotoxin-producing Escherichia coli O157 in beef burgers linked to an outbreak of diarrhoea, haemorrhagic colitis and haemolytic uremic syndrome in Britain. Lett. App. Microbiol. 19: 304-7.

115. Wilson, I. G. 1997. Inhibition and facilitation of nucleic acid amplification. Appl. Environ. Microbiol. 63:3741-51.

116. Yu, J. and Kaper, J. B 1992. Cloning and characterization of the eae gene of enterohemorrhagic Escherichia coli O157:H7. Mol. Microbiol. 6: 411-7.

117. Yaron. S and Matthews, K. R 2002. A reverse transcriptase-polymerase chain reaction assay for detection of viable Escherchia coli O157:H7 investigation of specific target genes. J. Appl. Micrbiol. 92:663-643.

Various embodiments of the present invention having been thus described in detail by way of example, it will be apparent to those skilled in the art that variations and modifications may be made without departing from the invention. The invention includes all such variations and modifications as fall within the scope of the appended claims.

What is claimed is:

1. A method useful to detect a pathogenic microbe, the method comprising the step of subjecting DNA extracted from said microbe or a cDNA equivalent thereof, to a polymerase chain reaction comprising primers adapted to produce a detectable amplicon from a gene responsible for the pathogenicity of said microbe, and measuring in real time the accumulation of said amplicon during said reaction.

2. The method according to claim 1, wherein the polymerase chain reaction is performed in the presence of probe that selectively binds said amplicon and incorporates a label detectable upon reaction of the probe with a 5′ nuclease.

3. The method according to claim 1, for the detection of at least two different pathogenic microbes in a given sample, the method comprising the step of subjecting a sample comprising DNA extracted from said microbes, or a cDNA equivalent thereof, to a polymerase chain reaction comprising primers adapted to produce at least one detectable amplicon from at least one gene of each pathogenic microbe in said sample, and then measuring in real time the accumulation of said amplicons during the reaction.

4. The method according to claim 1, for the detection of at least one pathogenic microbe selected from total coliforms, E. coli, E. coli O157:H7, toxigenic M. aeruginosa, G. lamblia, and C. parvum.

5. An amplicon having a nucleotide sequence selected from the coding sequence:
(a) the region spanning residues 2574-2895 of the lacZ gene of E. coli;
(b) the region spanning residues 2673-2759 of the eaeA gene of E. coli O157:H7;
(c) the region spanning residues 1438-1559 of the mcyA gene of Microcystis aeruginosa;
(d) the region spanning residues 222-296 of the β-giardin gene of G. lamblia;
(e) the region spanning residues 411-485 of the β-giardin gene of G. lamblia; and
(f) the region spanning residues 583-733 of the COWP gene of C. parvum.

6. An oligonucleotide probe that binds selectively to an amplicon defined in claim 5.

7. An oligonucleotide probe according to claim 6, bearing a fluorophore detectable upon reaction with a 5′ nuclease.

8. An oligonucleotide probe having a nucleotide sequence selected from SEQ ID Nos. 3, 6, 9, 12, 15 and 18.

9. An oligonucleotide primer adapted to amplify an amplicon according to claim 5.

10. An oligonucleotide primer according to claim 9, having a nucleotide sequence selected from SEQ ID NOs. 1, 2, 4, 5, 7, 8, 10, 11, 13, 14, 16 and 17.

11. A method for detecting total coliforms including E. coli in a given sample, comprising the step of subjecting DNA extracted therefrom to a polymerase chain reaction incorporating primers having SEQ ID NOs 4 and 5, and a probe having SEQ ID NO. 6.

12. A method for detecting E. coli O157:H7 in a given sample, comprising the step of subjecting DNA extracted therefrom to a polymerase chain reaction incorporating primers having SEQ ID NOs 1 and 2, and a probe having SEQ ID NO. 3.

13. A method for detecting M. aeuroginosa in given sample, comprising the step of subjecting DNA extracted therefrom to a polymerase chain reaction incorporating primers having SEQ ID NOs 7 and 8, and a probe having SEQ ID NO. 9.

14. A method for detecting G. lamblia in a given sample, comprising the step of subjecting DNA extracted therefrom to a polymerase chain reaction incorporating either (A) primers having SEQ ID NOs 10 and 11, and a probe having SEQ ID NO. 12, or (B) pimers having SEQ ID NOs 13 and 14, and a probe having SEQ ID NO. 15.

15. A method for detecting C. parvum in given sample, comprising the step of subjecting DNA extracted therefrom to a polymerase chain reaction incorporating primers having SEQ ID NOs 16 and 17, and a probe having SEQ ID NO. 18.

16. A method for discriminating between microbes G. lamblia and G. muris, comprising the step of subjecting DNA extracted from a selected one of said organisms to first and second polymerase chain reactions adapted to generate the amplicons of claim 5(d) and claim 5(e) respectively, and then identifying the microbe as G. lamblia in the case where both amplicon(s) are detected.

17. A method for discriminating between the assemblage A and assemblage B genotypes of G. lamblia, comprising the step of subjecting DNA extracted therefrom to first and second polymerase chain reactions using (1) the primer and probes of SEQ ID NO.s 13, 14 and 15, and (2) the primer and probe sets of SEQ ID NO.s 19, 20 and 21, and then identifying the genotype as assemblage A in the case where the primer and probe set (1) produces a detectable amplicon.

18. A method according to claim 1, wherein the extracted DNA is treated, prior to amplification, with at least one agent to reduce inhibitors of a polymerase chain reaction.

19. The method according to claim 18, wherein the agent includes a binding agent selected from an ion chelator and a protein scavenger.

20. A method according to claim 1, adapted for detection of DNA extracted only from viable cells.


SEQUENCE LISTING (21 sequences)

SEQ ID NO: 1 - DNA, 28 bp, Escherichia coli 0157:H7
  ref <308>: DATABASE ACCESSION NUMBER: GenBank X60439
  ref <309>: DATABASE ENTRY DATE: 1992-02-28
  ref <313>: RELEVANT RESIDUES: (2673)..(2700)
aataactgcttggattaaacagacatct

SEQ ID NO: 2 - DNA, 26 bp, Escherichia coli 0157:H7
  ref <308>: DATABASE ACCESSION NUMBER: GenBank X60439
  ref <309>: DATABASE ENTRY DATE: 1992-02-28
  ref <313>: RELEVANT RESIDUES: (2734)..(2759)
ggaagagggttttgtgttattaggtt

SEQ ID NO: 3 - DNA, 29 bp, Escherichia coli 0157:H7
  ref <308>: DATABASE ACCESSION NUMBER: GenBank X60439
  ref <309>: DATABASE ENTRY DATE: 1992-02-28
  ref <313>: RELEVANT RESIDUES: (2703)..(2731)
aagtgcttgatactccagaacgctgctca

SEQ ID NO: 4 - DNA, 22 bp, Escherichia coli
  ref <308>: DATABASE ACCESSION NUMBER: v00296
  ref <309>: DATABASE ENTRY DATE: 1996-03-06
  ref <313>: RELEVANT RESIDUES: (2754)..(2775)
ggatctgccattgtcagacatg

SEQ ID NO: 5 - DNA, 22 bp, Escherichia coli
  ref <308>: DATABASE ACCESSION NUMBER: v00296
  ref <309>: DATABASE ENTRY DATE: 1996-03-06
  ref <313>: RELEVANT RESIDUES: (2874)..(2895)
ctgttgactgtagcggctgatg

SEQ ID NO: 6 - DNA, 23 bp, Escherichia coli
  ref <308>: DATABASE ACCESSION NUMBER: v00296
  ref <309>: DATABASE ENTRY DATE: 1996-03-06
  ref <313>: RELEVANT RESIDUES: (2778)..(2800)
taccccgtacgtcttcccgagcg

SEQ ID NO: 7 - DNA, 20 bp, Microcystis cf. aeruginosa
  ref <308>: DATABASE ACCESSION NUMBER: AB019578
  ref <309>: DATABASE ENTRY DATE: 1999-09-15
  ref <313>: RELEVANT RESIDUES: (1438)..(1457)
cgaccgaggaatttcaagct

SEQ ID NO: 8 - DNA, 24 bp, Microcystis cf. aeruginosa
  ref <308>: DATABASE ACCESSION NUMBER: AB019578
  ref <309>: DATABASE ENTRY DATE: 1999-09-15
  ref <313>: RELEVANT RESIDUES: (1536)..(1559)
agtatccgaccaagttacccaaac

SEQ ID NO: 9 - DNA, 31 bp, Microcystis cf. aeruginosa
  ref <308>: DATABASE ACCESSION NUMBER: AB019578
  ref <309>: DATABASE ENTRY DATE: 1999-09-15
  ref <313>: RELEVANT RESIDUES: (1459)..(1489)
ttaaatcggaaattatcccagaaaatgccgt

SEQ ID NO: 10 - DNA, 18 bp, Giardia lamblia
  ref <308>: DATABASE ACCESSION NUMBER: M36728
  ref <309>: DATABASE ENTRY DATE: 1994-04-14
  ref <313>: RELEVANT RESIDUES: (222)..(239)
catccgcgaggaggtcaa

SEQ ID NO: 11 - DNA, 19 bp, Giardia lamblia
  ref <308>: DATABASE ACCESSION NUMBER: M36728
  ref <309>: DATABASE ENTRY DATE: 1994-04-14
  ref <313>: RELEVANT RESIDUES: (278)..(296)
gcagccatggtgtcgatct

SEQ ID NO: 12 - DNA, 28 bp, Giardia lamblia
  ref <308>: DATABASE ACCESSION NUMBER: M36728
  ref <309>: DATABASE ENTRY DATE: 1994-04-14
  ref <313>: RELEVANT RESIDUES: (241)..(268)
aagtccgccgacaacatgtacctaacga

SEQ ID NO: 13 - DNA, 22 bp, Giardia lamblia Portland-1
  ref <308>: DATABASE ACCESSION NUMBER: M36728
  ref <309>: DATABASE ENTRY DATE: 1994-04-14
  ref <313>: RELEVANT RESIDUES: (411)..(432)
cctcaagagcctgaacgatctc

SEQ ID NO: 14 - DNA, 24 bp, Giardia lamblia Portland-1
  ref <308>: DATABASE ACCESSION NUMBER: M36728
  ref <309>: DATABASE ENTRY DATE: 1994-04-14
  ref <313>: RELEVANT RESIDUES: (462)..(485)
agctggtcgtacatcttcttcctt

SEQ ID NO: 15 - DNA, 22 bp, Giardia lamblia Portland-1
  ref <308>: DATABASE ACCESSION NUMBER: M36728
  ref <309>: DATABASE ENTRY DATE: 1994-04-14
  ref <313>: RELEVANT RESIDUES: (434)..(455)
ttctccgtggcaatgcccgtct

SEQ ID NO: 16 - DNA, 25 bp, Cryptosporidium parvum
  ref <308>: DATABASE ACCESSION NUMBER: Z22537
  ref <309>: DATABASE ENTRY DATE: 1995-08-29
  ref <313>: RELEVANT RESIDUES: (583)..(607)
caaattgataccgtttgtccttctg

SEQ ID NO: 17 - DNA, 23 bp, Cryptosporidium parvum
  ref <308>: DATABASE ACCESSION NUMBER: Z22537
  ref <309>: DATABASE ENTRY DATE: 1995-08-29
  ref <313>: RELEVANT RESIDUES: (711)..(733)
ggcatgtcgattctaattcagct

SEQ ID NO: 18 - DNA, 31 bp, Cryptosporidium parvum
  ref <308>: DATABASE ACCESSION NUMBER: Z22537
  ref <309>: DATABASE ENTRY DATE: 1995-08-29
  ref <313>: RELEVANT RESIDUES: (672)..(702)
tgccatacattgttgtcctgacaaattgaat

SEQ ID NO: 19 - DNA, 22 bp, Giardia lamblia Portland 1
cctcaagagcctgaacgacctc

SEQ ID NO: 20 - DNA, 24 bp, Giardia lamblia Portland-1
agctggtcatacatcttcttcctc

SEQ ID NO: 21 - DNA, 22 bp, Giardia lamblia Portlan-1
ttctccgtgggaatgcctgtct